(19) United States
(12) Patent Application Publication
(10) Pub. No.: US 2019/0153475 A1
(43) Pub. Date: May 23, 2019

(54) GENE CASSETTE FOR HOMOLOGOUS RECOMBINATION KNOCK-OUT IN YEAST CELLS

(71) Applicant: EVONIK DEGUSSA (CHINA) CO., LTD, Beijing (CN)

(72) Inventors: Xianzhong Chen, Wuxi (CN); Lihua Zhang, Xinyang (CN); Zheng Xiang, Wuxi (CN); Wei Shen, Wuxi (CN); Li Li, Shanghai (CN); Markus Pötter, Münster (DE)

(73) Assignee: Evonik Degussa (China) Co., Ltd., Shanghai (CN)

(21) Appl. No.: 16/061,681

(22) PCT Filed: Dec. 17, 2015
(86) PCT No.: PCT/CN2015/097675

§ 371 Date: Jun. 13, 2018

(51) Int. Cl. C12N 15/90; C12R 1/74; C12N 15/81; C12N 15/10

(52) U.S. Cl. CPC C12N 15/905; C12N 15/102; C12N 15/81; C12R 1/74

(57) ABSTRACT

There is provided a gene cassette for disruption of at least one target gene in a yeast cell, wherein the gene cassette comprises:
(d) a URA3 gene capable of being used as a marker gene;
(e) at least one gene disruption auxiliary (gda) sequence; and
(f) an upstream and a downstream sequences of the target gene,
   wherein the gda sequence is at least 300 to 600 bp in length and selected from within the nucleotide sequence of SEQ ID NO:39.

### CROSS REFERENCE TO RELATED APPLICATIONS

This application is a 35 U.S.C. § 371 U.S. national phase entry of International Application No. PCT/CN2015/097675 having an international filing date of Dec. 17, 2015, of which is incorporated herein by reference in its entirety.

### FIELD

The present invention relates to a gene cassette for use in disrupting a target gene in at least one yeast cell. In particular, the gene marker may comprise a reusable selection marker and at least one further disruption sequence that may be used to disrupt the expression of at least one target gene.

### BACKGROUND

Yeasts are well known for use in the world of genetic engineering for producing specific compounds and/or compositions. In particular, yeast strains may be genetically modified to disrupt the expression of specific genes thus enabling these genetically modified strains to be useful in the production of desired compounds and/or compositions. The modification of a yeast strain makes it possible to obtain yeasts with different or improved properties, which can be used in many possible applications, among which breadmaking, the food industry, health, the production of compounds, for example alcohol, the production of yeast extracts.

For example, some yeast species such as S. cerevisiae are known to ferment hexose sugars predominantly into ethanol, rather than the mixtures of products typically produced by bacteria. Some yeasts have other characteristics that make them good candidates for various types of fermentation processes, such as resistance to low pH environments, resistance to certain fermentation co-products such as acetic acid and furfural, and resistance to ethanol itself. Yeast cells thus make good targets for genetic manipulation resulting in genetically modified cells with desired characteristics.

In another example, C. tropicalis is increasingly being used in the fermentation industry. For example, C. tropicalis, by means of the highly-efficient intracellular β-oxidation component of the ω-oxidation pathway, can be used to produce long-chain dicarboxylic acids (DCA) using alkanes and fatty acids as the sole carbon source and energy source. These DCAs are used in a broad range of applications in the chemical and pharmaceutical industries. At present, C. tropicalis is the strain most commonly used in the fermentation industry to produce diacids. C. tropicalis is also commonly used in xylitol production. C. tropicalis has also been found to show a number of benefits in the area of environmental protection, particularly in the biological treatment of industrial and agricultural wastewater, not only showing a capacity to break down organic liquid waste that is readily biodegradable, but simultaneously producing single cell protein, both reducing environmental pollution and making waste profitable by producing valuable products. However, C. tropicalis being a diploid yeast, does not have a sexual reproduction stage, reproducing only asexually. C. tropicalis has numerous other physiological properties that make it different from S. cerevisiae and thus genetic manipulation of C. tropicalis is a lot more complicated. In particular, the metabolic network involved in C. tropicalis is highly complex and there have been numerous drawbacks in its direct application in industrial production. For this reason, there appears new methods of strain improvement by metabolic engineering of C. tropicalis (Haas L, Cregg J et al., 1990). Using this transformation system, Picataggio et al. carried out sequential disruption of the POX4 and POX5 genes to produce a strain in which the β-oxidation pathway was blocked, establishing a sequential gene disruption system (Picataggio S, Deanda K et al. 1991). In order to reuse the URA3 marker, they screened for spontaneous mutations or used molecular methods to disrupt the introduced URA3 gene. Gao Hong et al., on the other hand, on the basis of using hygromycin B's resistance to disrupt single-copy CAT gene, used G418 resistance as a second selection marker and constructed a corresponding disruption cassette, and achieved CAT gene double-copy destruction (Gao Hong, 2005). However, there are still numerous problems associated with using antibiotic resistance as a selection marker in C. tropicalis as some strains fair poorly with antibiotics and thus this imposes limitations on gene transformation and molecular improvement. Further, C. tropicalis has the characteristic property of translating the CTG codon (ordinarily translated as leucine) as serine, and this further increases the difficultly of using an exogenous resistance gene. Also, multiple resistance markers are often required in the gene disruption of diploid yeasts to complete multiple copy disruption, which further limit the use of resistance markers. The genetic manipulation of C. tropicalis for these reasons and more are known to be complicated.

In the ura-blaster sequential gene disruption system of Alani et al. (Alani E, Cao L et al. 1987), a hisG sequence is inserted on each side of the URA3 gene with the same direction, this makes the entire gene disruption cassette too large, and it is therefore every difficult to effectively amplify the entire disruption cassette by PCR (R. Bryce Wilson, Dana Davis et al. 2000).

The lack of efficiency and simplicity of the currently available methods for genetically modifying yeast cells make the process of producing genetically modified yeast cells complicated. Thus, there is still a need to provide novel methods for obtaining improved strains of yeast, these methods being faster and simple to carry out and allowing a more efficient selection of the yeast strains having the desired improvements.

### SUMMARY

The present invention attempts to solve the problems above by providing a means of disrupting a target gene in a yeast cell. In particular, the means of disrupting the yeast cell comprises a nucleotide sequence that comprises a marker gene, a short DNA fragment of the marker gene including the upstream and downstream sequence and an upstream and downstream sequence of the target gene. Even more in particular, the marker gene may be the URA3 gene and the short DNA fragment of the marker gene may be about 300 to 600 bp in length selected from the sequence of −420 bp to +1158 bp of the URA3 gene (i.e. 420 bp upstream of the start codon of URA3 and 354 bp downstream of the stop codon of URA3).

According to one aspect of the present invention, there is provided a gene cassette for disruption of at least one target gene in a yeast cell, wherein the gene cassette comprises:
(a) a URA3 gene capable of being used as a marker gene;
(b) at least one gene disruption auxiliary (gda) sequence; and
(C) an upstream and a downstream sequence of the target gene,
   wherein the gda sequence is at least 300 to 600 bp in length and selected from within the nucleotide sequence of SEQ ID NO:39 and variants thereof. In particular, the gda sequence may be a URA3 fragment.

### DETAILED DESCRIPTION

The gene cassette according to any aspect of the present invention makes smart use of the fact that the gda sequence is a fragment of the URA3 gene, allowing highly effective loss of the URA3 gene during chromosomal replication of yeast, in one example C. tropicalis, and therefore can effectively reuse the URA3 marker gene. Compared with the conventional gene disruption cassette CAT1-hisG-URA3-hisG-CAT1, which uses the hisG sequence of Salmonella typhimurium inserted in the same direction on both sides of the URA3 marker gene, the gene cassette according to any aspect of the present invention uses a fairly short gda sequence and requires insertion of only one gda sequence, which sharply reduces the length of the disruption cassette, making it convenient for use in molecular biology operations such as PCR. Furthermore, the transformation/recombination efficiency of the disruption cassette may be considered to be markedly superior, resulting in a further substantial improvement in the overall efficiency of C. tropicalis gene disruption.

A yeast selection marker gene may be selected from a known gene capable of being selected for: such genes include but are not limited to genes encoding auxotrophic markers, such as LEU2, HIS3, TRP1, URA3, ADE2 and LYS2. Alternatively, genes encoding a protein conferring drug resistance on a host cell can be used as a yeast selection marker. Such genes include, but are not limited to CAN1 and CYH2. In particular, “a yeast Selectable Marker” as used herein refers to a genetic element encoding a protein that when expressed in yeast that enables selection of the yeast cell by the presence of the protein. Thus, any yeast cell that contains and expresses a yeast selectable marker can be differentiated from otherwise similar yeast cells that do not contain and express the marker. Examples include TRP1, HIS3, URA3, and LEU2. In particular, the yeast selection marker used according to any aspect of the present invention may be URA3 gene. DNA coding for orotidine-5′-phosphate decarboxylase (EC4.1.1.23) (URA3 gene) may be used as a marker gene according to any aspect of the present invention. This enzyme is an essential enzyme in pyrimidine biosynthesis in several yeast strains. This selection marker used according to any aspect of the present invention may be reusable. The URA3 gene sequence or URA3 gene refers to a cassette comprising an upstream regulatory sequence, a coding region, and a downstream regulatory sequence. URA3 gene represents a gene fragment comprising 5′ untranslated region containing a promoter region, a region coding for orotidine-5′-phosphate decarboxylase (EC4.1.1.23), and 3′ untranslated region containing a terminator region. In one example, the base sequence of URA3 gene may be from Candida maltosa which may be disclosed as D12720 in the NCBI genebank database. In another example, the URA3 gene may be from C. tropicalis STXX 20336. In particular, the URA3 gene according to any aspect of the present invention may comprise the sequence of SEQ ID NO: 3. It may be considered to be advantageous to use URA3 gene as a selection marker for its ability to be used repeatedly as a convenient selectable marker. URA3, an auxotrophic marker, may be convenient for the introduction of knock-out mutations. The chosen marker such as URA3 may also act as both a selectable and a counter-selectable marker to permit one to first select for and then eliminate that marker in a subsequent selection step. The URA3 gene pop-out efficiency may be considered stable.

Use of these genes as markers is restricted to host strains that are auxotrophic for the nutrient in question due to the absence of a functional chromosomal copy of the marker gene. Unless transformed to prototrophy with a functional allele of the marker gene, auxotrophic yeast strains can be propagated only in media that contain the appropriate growth factor(s). This nutritional complementation may be achieved either by including the growth factors in defined synthetic media or by using complex medium components (e.g., yeast extract and peptone) that are rich in the relevant growth factors.

The gene cassette according to any aspect of the present invention further comprises at least one URA3 gene fragment. This gene fragment may be called a gene disruption auxiliary (gda) sequence. It was surprisingly found according to any aspect of the present invention that when a gene cassette with:
(a) URA3 as the marker gene;
(b) a gda sequence selected from a URA3 gene having its upstream site at −420 bp (i.e., 420 bp upstream of the start codon) and having its downstream site at +1158 bp (i.e. 354 bp downstream of the stop codon), and
(c) a homology arm of the target gene at the each end of the cassette
   can effectively delete genes in yeast strains. Further, the URA3 gene selection marker can be repeatedly used. According to any aspect of the present invention, where the position of the a sequence is expressed as −n bp and/or +m bp (where n and m are integers), the positional standards are as follows: the position of A in the start codon ATG of the URA3 gene in the coding region is designated as +1 bp, and position of the first base pair upstream of the start codon ATG (i.e., the first base pair adjacent to the left side of A) is designated as −1 bp. Therefore, the position of then base pair upstream of the start codon ATG will be designated −nbp (also referred to as nbp upstream of start codon or −nbp), the position of T in the start codon ATG is designated as +2 bp, and under the downstream of the start codon ATG, with the A of the ATG designating as the first base (pair), the position of the m base (pair) is designated as +mbp (also referred to as mbp downstream of the start codon or +mbp). This method of numbering is illustrated in FIG. 3.

In particular, the gda sequence may be selected from within SEQ ID NO:39.

In particular, the size of the gda sequence may be 100 to 600 bp. The length of the gda sequence may be varied. The shorter the sequence, the cheaper the cost of making the gene cassettes as less raw materials (i.e. medium, dNTPs etc.) need to be used. The size of the gda sequence may be reduced to also obtain a cassette of which may be smaller than the cassettes known in the art for the same purpose. The gene cassette according to any aspect of the present invention may show substantially improved transformation or recombination efficiency compared with conventional gene disruption cassettes using hisG sequences. This is confirmed by the examples provided. Even more in particular, the size of the gda sequence may be 300 to 500 bp according to any aspect of the present invention. It was also surprisingly found that when the length of the gda sequence of the gene cassette according to any aspect of the present invention was 300-500 bp, the URA3 gene pop-out efficiency after transformation into a yeast strain, for example, uracil auxotroph C. tropicalis may be considerably increased. In particular, the URA3 gene pop-out efficiency after transformation into a yeast strain may be comparable to that of conventional hisG gene disruption cassettes. Since the transformation efficiency of the gene disruption cassette according to any aspect of the present invention may be remarkably higher than that of conventional gene disruption cassettes, significantly improved overall gene disruption efficiency of yeast strains, in particular C. tropicalis may be achieved. In one example, the gda sequence may be about 100, 150, 200, 250, 300, 350, 400, 450, 500, 550 or 600 bp in length. In another example, the gda sequence according to any aspect of the present invention may be 100-600 bp, 150-600, 200-600, 250-600, 300-600, 350-600, 400-600, 100-550, 100-500, 100-450, 100-400, 100-350, 100-300, 150-600, 150-550, 150-500, 150-450, 150-400, 150-350, 200-550, 200-500, 200-450, 200-400, 250-550, 250-500, 250-450, 250-400, 250-350, 300-550, 300-500, 300-450, 300-400 bp, 302-600 bp, 302-500 bp, 302-488 bp, 305-488 bp or the like in length. A skilled person may be capable of identifying the length of gda sequence that may be suitable in each case depending on the target sequence that is to be disrupted by the gene cassette. In particular, the size of the gda sequence may be 200 to 500 bp. More in particular, the gda sequence may be 300 to 500 bp. In one example, the length of the gda sequence may be selected from the group consisting of 143, 245, 302, 305, 324, 325 and 488 bp in length. In particular, the length of the gda sequence may be 324 or 325 bp in length.

In another example, there may be more than one gda sequence in the gene cassette according to any aspect of the present invention. In particular, there may be two or three gda sequences in the gene cassette. More in particular, the gene cassette according to any aspect of the present invention comprises only one gda sequence so that the cassette formed will be shorter and thus easier to make and use.

The fact that the gda sequences can be varied in length may be considered advantageous for the formation of the gene cassette according to any aspect of the present invention. In particular, it may be very convenient for disruption of two adjacent genes on the same chromosome (such as the C. tropicalis POX4 and POX2 genes), and this establishes a basis for the further use of molecular biological techniques in researching the C. tropicalis genes.

Further, when the length of the gda sequence of the gene cassette according to any aspect of the present invention is from 300 bp to 500 bp, the URA3 gene pop-out efficiency after the gene cassette according to any aspect of the present invention is transformed into C. tropicalis is sharply increased, therefore further increasing the overall efficiency of C. tropicalis gene disruption. Other advantages according to any aspect of the present invention would be apparent for a person skilled in the art after reading the present specification.

According to any aspect of the present invention, the gda sequence may be selected from within the nucleotide sequence of SEQ ID NO:39 and variants thereof. In particular, the gda sequence according to any aspect of the present invention may be 100-600 bp, 100-500 bp, 200-500 bp, 300-500 bp selected from within the nucleotide sequence of SEQ ID NO:39 and variants thereof of URA3 gene sequence.

The term ‘variants’ may refer to amino acid or nucleic acid sequences, respectively, that are at least 70, 75, 80, 85, 90, 92, 94, 95, 96, 97, 98, 99 or 99.5% identical to the reference amino acid or nucleic acid sequence, wherein preferably amino acids other than those essential for the function, for example the catalytic activity of a protein, or the fold or structure of a molecule are deleted, substituted or replaced by insertions or essential amino acids are replaced in a conservative manner to the effect that the biological activity of the reference sequence or a molecule derived therefrom is preserved. The state of the art comprises algorithms that may be used to align two given nucleic acid or amino acid sequences and to calculate the degree of identity, see Arthur Lesk (2008), Introduction to bioinformatics, 3^{rd} edition, Thompson et al., Nucleic Acids Research 22, 4637-4680, 1994, and Katoh et al., Genome Information, 16(1), 22-33, 2005. Such variants may be prepared by introducing deletions, insertions or substitutions in amino acid or nucleic acid sequences as well as fusions comprising such macromolecules or variants thereof. In one example, the term “variant”, with regard to amino acid sequence, comprises, in addition to the above sequence identity, amino acid sequences that comprise one or more conservative amino acid changes with respect to the respective reference or wild type sequence or comprises nucleic acid sequences encoding amino acid sequences that comprise one or more conservative amino acid changes. In another example, the term “variant” of an amino acid sequence or nucleic acid sequence comprises, in addition to the above degree of sequence identity, any active portion and/or fragment of the amino acid sequence or nucleic acid sequence, respectively, or any nucleic acid sequence encoding an active portion and/or fragment of an amino acid sequence. In particular, the term “active portion”, as used herein, refers to an amino acid sequence or a nucleic acid sequence, which is less than the full length amino acid sequence or codes for less than the full length amino acid sequence, respectively, wherein the amino acid sequence or the amino acid sequence encoded, respectively retains at least some of its essential biological activity. For example an active portion and/or fragment of a protease is capable of hydrolysing peptide bonds in polypeptides. In one example, the term “retains at least some of its essential biological activity”, as used herein, means that the amino acid sequence in question has a biological activity exceeding and distinct from the background activity and the kinetic parameters characterising said activity, more specifically k_{cat} and K_{M}, are preferably within 3, more preferably 2, most preferably one order of magnitude of the values displayed by the reference molecule with respect to a specific substrate. In one example the term “variant” of a nucleic acid comprises nucleic acids the complementary strand of which hybridises, preferably under stringent conditions, to the reference or wild type nucleic acid. Examples of variants of URA3 and/or SEQ ID NO: 3 may include but are not limited to AF040702.1, GQ268324.1, JX100416.1, AY033329.1, EU288194.1, GQ268324.1, AF321098.1, AF109400.1, U40564.1, K02207.1 and the like.

In another example, the gda sequence according to any aspect of the present invention may be 100-600 bp, 100-500 bp, 200-500 bp, 300-500 bp selected from within the nucleotide sequence of −420 bp to +318 bp of the URA3 gene. (from 420 bp upstream to 318 bp downstream of the start codon) In particular, the gda sequence may be selected from within SEQ ID NO:40. In another example, the gda sequence according to any aspect of the present invention may be 100-600 bp, 100-500 bp, 200-500 bp, 300-500 bp selected from within the nucleotide sequence of +533 bp to +1158 bp of the URA3 gene (from 272 bp upstream to 354 by downstream of the stop codon). In particular, the gda sequence may be selected from within SEQ ID NO:41. In a further example, the gda sequence according to any aspect of the present invention may be 100-600 bp, 100-500 bp, 200-500 bp, 300-500 bp selected from within the nucleotide sequence of +804 bp to +1158 bp of the URA3 gene (from the stop codon to 354 bp downstream of the stop codon). In particular, the gda sequence may be selected from within SEQ ID NO:42. In yet another example, the gda sequence according to any aspect of the present invention may be 100-600 bp, 100-500 bp, 200-500 bp, 300-500 bp selected from within the nucleotide sequence of 420 bp upstream of the start codon of URA3 gene coding region. In particular, the gda sequence may be selected from within SEQ ID NO:43. The positions of the different starting and ending points of part of the URA3 gene when SEQ ID NO:3 is used is shown in FIG. 4.

In one example, the upstream site of the gda sequence is located in the coding region of URA3 gene, and the downstream site of the gda sequence is located in a region between the stop codon of the coding region of URA3 gene and 354 bp downstream of the stop codon of the coding region of URA3 gene e.g. it has the nucleotide sequence shown in SEQ ID NO. 6.

In another example, the gda sequence according to any aspect of the invention may be from the URA3 gene coding region, e.g. has the nucleotide sequence shown in SEQ ID NO: 27, or the gda sequence is a sequence having the URA3 gene stop codon as its downstream site.

In yet another example, the gene cassette according to any aspect of the present invention comprises a gda sequence selected from the group consisting of SEQ ID NOs: 18, 16, 27, 24, 14, 21, and 6. The gda sequence may comprise sequence identity of at least 50% to any one of the sequences selected from the group consisting of SEQ ID NOs: 18, 16, 27, 24, 14, 21, and 6. Even more in particular, The gda sequence according to any aspect of the present invention may comprise sequence identity of at least 50% to the nucleotide sequences of SEQ ID NOs: 16, 14 or 21. More in particular, gda sequence may comprise a nucleotide with sequence identity of at least 50, 60, 65, 70, 75, 80, 85, 90, 91, 94, 95, 98, 99, 99.5 or 100% to a nucleotide selected from the group consisting of SEQ ID NOs: 16, 14 or 21.

In one example, the gda sequence according to an aspect of the present invention may be directly linked to the URA3 gene. In particular, the gda sequence may be inserted into a suitable site within the cassette using restriction enzymes. Consequently, the gda sequence may be directly linked to the URA3 gene in the cassette according to any aspect of the present invention and there may be no linker sequences connecting the gda to the URA3 gene. This makes the process of producing the cassette according to any aspect of the present invention easy and convenient. The gene cassette according to any aspect of the present invention may further comprise an upstream and a downstream sequence of a target gene which the gene cassette may be capable of disrupting the expression of in the yeast strain. The gene cassette may comprise regions that are highly identical to (i.e. identities score of 80% or more, preferably 95% or more and most preferably 100%) to the upstream (5′-) and downstream (3′-) flanks of the target gene. Either or both of these regions may include a portion of the coding region of the target gene as well as a portion or all of the respective promoter or terminator regions. In one example, the gda sequence and the URA3 marker may reside between the regions that are highly identical to the upstream and downstream flanks of the target gene. Any native gene of the yeast strain used may serve as targets for insertion of the gene cassette according to any aspect of the present invention.

Successful transformants can be selected for in known manner, by taking advantage of the attributes contributed by the marker gene, or by other characteristics (such as ability to produce lactic acid, inability to produce ethanol, or ability to grow on specific substrates) contributed by the inserted genes. Screening can be performed by PCR or Southern analysis to confirm that the desired insertions and deletions have taken place, to confirm copy number and to identify the point of integration of genes into the host cell's genome. Activity of the enzyme encoded by the inserted gene and/or lack of activity of enzyme encoded by the deleted gene can be confirmed using known assay methods.

According to any aspect of the present invention, a “target gene”, as used herein, refers to a gene the silencing or disruption of which causes a decreased growth, development, reproduction or survival of a pathogenic yeast. In one example, the partial or complete silencing of an essential gene of a yeast results in significant yeast mortality or significant yeast control when such gene is silenced as compared to control yeast applied with a nucleotide sequence targeting a non-essential gene or a gene not naturally expressed in the yeast. In another example, the target gene used may be pyruvate decarboxylase gene and/or CAT gene.

The gene cassette according to any aspect of the present invention, may comprise upstream and downstream sequences of the target gene. ‘Arms of Homology’ as used herein, refers to a pair of DNA segments that is present in the gene cassette according to any aspect of the present invention; these segments are homologous to two portions of the target gene. Because of this homology, the arms of homology will be able to undergo homologous recombination with the target gene. The arms of homology are at least 50 bp in length to allow for appreciable rates of homologous recombination to occur in yeast cells. More in particular, the arms of homology may be ≥40, 55, 60, 65, 70, 80, 90, 100 in length. The ‘arms of homology’ may also be referred to as the upward and downward sequences of the target gene that flank the target gene. The two sequences of DNA (upward and downstream sequences) homologous to the genomic DNA flank the DNA gene sequence (target gene) to be deleted/disrupted. These flanking sequences are termed arms of homology. In particular, these arms of homology are substantially isogenic for the corresponding flanking sequences in the target gene on the yeast cell. The use of DNA that is substantially isogenic to the target gene helps assure high efficiency of recombination with the target sequences. The gene cassette according to any aspect of the present invention, includes at least a positive selection marker (URA3) within the arms of homology to enable the scoring of recombination. Such positive selection markers can confer a phenotype not normally exhibited by wild-type yeast; for example, resistance to a substance normally toxic to the target cells. In another example, the cassette according to any aspect of the present invention also includes one or more negative selection markers outside the arms of homology to facilitate identification of proper homologous recombinants. U.S. Pat. No. 5,464,764 describes the use of such “positive-negative” selection methods. Upon successful gene-targeting and homologous recombination, the positive selection marker is incorporated into the genome within the arms of homology in place of the targeted gene segment, while the negative selection marker is excluded. Thus, to enrich for homologous recombinants, gene-targeted cells are grown in culture medium containing the appropriate positive and negative selective compounds.

In one example, the arms of homology may be ≥17, 18, 19, 20, 21, 22, 23, 24, 25, 26, 27, 28, 29, 30, 31, 32, 33, 34, 35, 40, 45, in particular, 50, 55, 60, 65, 70, 75, 80, 85, 90, 95, 100, 110, 120, 150, 200, 300, 400, 500, 600, 700, 800, 900 or 1000 contiguous nucleotides of the target gene of one of the nucleotide strands. The upstream and downstream sequences may not refer to the exact sequence of the target gene but the region flanking the start and stop codon of the target gene. In another example, the start and stop codon may be part of the upstream and downstream sequences respectively. In a further example, the upstream and downstream sequences of the target gene may each be ≥50 bp in length.

Any yeast cell may be used according to any aspect of the present invention. In particular, the yeast cell may be selected from the group consisting of Candida albicans, Candida tropicalis, Candida parapsilopsis, Candida krusei, Cryptococcus neoformans, Hansenular polymorpha, Issatchenkia orientalis, Kluyverei lactis, Kluyveromyces lactis, Kluyveromyces marxianus, Pichia pastoris, Saccharomyces cerevisiae Schizosaccharomyces pombe, and Yarrowia lipolytica. In particular, the yeast cell may be C. tropicalis. An important area of application of auxotrophic yeast strains and the corresponding marker genes is the stable maintenance of expression vectors for the high-level production of native or heterologous proteins. In one example, the yeast cell used according to any aspect of the present invention may be uracil auxotrophic C. tropicalis. In one particular example, the C. tropicalis uracil auxotroph strain used according to any aspect of the present invention may be screened and obtained after physical or chemical mutagenesis of C. tropicalis ATTC 20336.

According to a further aspect of the present invention, there is provided a method of disrupting the expression of at least one target gene in at least one yeast cell, the method comprising the transforming of the yeast cell with the gene cassette according to any aspect of the present invention.

The method according to any aspect of the present invention may be suitable for two-copy and multiple gene disruption of C. tropicalis.

In one example, the construction method of the gene cassette according to any aspect of the present invention may comprise the following steps:
(1). Preparation of upstream and downstream sequences of the target gene: designing a promoter according to a known base sequence of a target gene, amplifying by PCR to obtain the upstream and downstream sequences of the target gene, or synthesizing the upstream and downstream sequences of the target gene according to a known sequence of a target gene; the length of the upstream and downstream sequences of the target gene being not less than 50 bp;
(2). Preparation of the URA3 marker gene: designing a promoter according to the URA3 sequence in the chromosome of C. tropicalis, e.g. C. tropicalis ATTC 20336, amplifying by PCR to obtain a URA3 gene of C. tropicalis comprising an upstream regulatory sequence, a coding region, and a downstream regulatory sequence;
(3). Preparation of gda sequence: designing a promoter according to the URA3 sequence in the chromosome of C. tropicalis, e.g. C. tropicalis ATTC 20336, amplifying by PCR to obtain the gda sequence, the gda sequence being derived from a URA3 gene coding region and/or regulatory sequence and having a length of 300-500 bp;
(4). Construction of the gene disruption cassette: linking the gda sequence obtained to upstream or downstream of the URA3 gene sequence to obtain a gda-URA3 or URA3-gda fragment; linking the sequences upstream and downstream of the target gene sequence to the two sides of gda-URA3 fragment respectively, thus obtaining the gene disruption cassette of the present invention.

The gene disruption cassette of the present invention may be represented as: upstream (or downstream) sequence of target gene-gda sequence-URA3 gene-downstream (or upstream) sequence of target gene, or upstream (or downstream) sequence of target gene-URA3 gene-gda sequence-downstream (or upstream) sequence of target gene.

A person skilled in the art may choose to construct gda-URA3 or URA3-gda fragment based on the selected gda sequence. For example, if a gda sequence is derived from the upstream sequence of the URA3 gene (including the upstream regulatory sequence or the N-terminal coding sequence of the coding region) and is to be inserted to 3′ end of URA3 gene, a URA3-gda fragment will be constructed, to facilitate subsequent gene combination and popping out of URA3 gene, i.e., popping out the fragment between the gda sequence and the corresponding source sequence of the gda sequence in URA3 gene.

According to another aspect of the present invention, there is provided a use of the gene cassette according to any aspect of the present invention in disruption of a target gene in a yeast cell, particularly a C. tropicalis cell, more particularly, in a uracil auxotroph C. tropicalis cell.

According to a further aspect of the present invention, there is provided a vector comprising the gene cassette according to any aspect of the present invention.

According to yet another aspect of the present invention, there is provided a cell comprising the gene cassette according to any aspect of the present invention.

According to a further aspect of the present invention, there is provided a multicellular organism comprising the gene cassette according to any aspect of the present invention.

In one example, the gene cassette according to any aspect of the present invention may be used in a method for deleting a target gene from the C. tropicalis strain, comprising the following steps:
(1) Transformation of the gene cassette according to any aspect of the present invention: transforming the gene cassette according to any aspect of the present invention into C. tropicalis cells, in particular, uracil auxotroph C. tropicalis cells by means of a lithium acetate or lithium chloride method, and applying the cells onto an MM culture plate to produce transformants;
(2) Identification of transformants: culturing single colonies of transformants on MM medium, extracting the chromosomal DNA and amplifying by PCR;
(3) Marker gene loss (or pop-out): culturing the C. tropicalis strain with transformation of the gene cassette in SM medium at 30° C. and 200 rpm until the microbial concentration reaches an appropriate level, centrifuging to collect the microbes, washing with sterile deionized water, applying onto a FOA plate, and culturing at 30° C.;
(4) Identification of marker gene loss: inoculating the grown single microbial colonies onto an SM plate and culturing, extracting the chromosomal DNA and identifying by PCR, and obtaining the mutant strain showing URA3 marker gene loss.

The mutant strain showing URA3 marker gene loss may be used as a host strain for a second round of gene disruption. In this method, culture media and formulations such as the following may be used: MM (yeast nitrogen base without amino acids & ammonium sulfate, YNB 6.7 g/L; glucose 20 g/1; (NH₄)₂SO₄ 10 g/L); SM (MM+uracil 60 mg/L); FOA culture medium (SM+5-fluoroorotic acid 2 g/L).

By means of the gene cassette according to any aspect of the present invention, the present invention provides a method for highly efficient deletion of the double-copy target gene of C. tropicalis. In one example, this method according to any aspect of the present invention may be used in the deletion of other target genes from C. tropicalis, including CAT genes and PDC genes. In another example, the gene cassette according to any aspect of the present invention may be used to delete two alleles in the same cell. For example, the gene cassette may be used to delete the two CAT alleles in C. tropicalis cells.

Sequencing of the target gene locus (for example CAT gene locus) of the strain after the marker gene pops out may be a means used to determine exactly where the loss of target gene takes place and to confirm the loss of target gene. For example, the loss may take place between the two CAT homology arms of a single CAT allele locus, and the fragment substituted by a gda sequence. Therefore, it may be demonstrated at molecular level that a target gene (such as a single copy of the CAT gene) may be successfully disrupted. Also, sequencing may also confirm two-copy target gene allele disruption.

In the gene disruption method for C. tropicalis based on a reusable selection marker used according to any aspect of the present invention, the principle of homologous recombination may be first used by conducting site-specific recombination with the gene cassette on the target gene locus of the auxotrophic strain, functionally destroying the target gene, then marker gene may be reused to screen strains with destroyed target gene. After this, 5-FOA selection pressure may be used to screen out mutant strains showing pop-out of the URA3 marker gene, and the mutant strains showing pop-out of the URA3 marker gene may be used to disrupt the second allele or other genes.

The gene cassette according to any aspect of the present invention, may have all three components (a), (b) and (c):
(a) a URA3 gene capable of being used as a marker gene;
(b) at least one gene disruption auxiliary (gda) sequence; and
(c) an upstream and a downstream sequences of the target gene.

Some examples of the order of the components (a), (b) and (c) are provided in FIG. 1. The gene cassette according to any aspect of the present invention may comprise components (a), (b) and (c) in any order. In one example, the gene cassette according to any aspect of the present invention may comprise a single (b) gda sequence. Component (b) may be located at the 3′ or 5′ end of (a) the URA3 gene. The upstream or the downstream sequence of component (c) may be located on the other end of (a) not bound to (b). It may be established that during pop-out of the URA3 marker gene, only the URA3 gene fragment between the gda sequence and the similar sequence within the URA3 gene may be popped out.

### BRIEF DESCRIPTION OF THE FIGURES

FIG. 1 are structure diagrams for the various gene disruption cassettes.

FIG. 2 is a flow chart of CAT gene disruption in the examples and indicates the binding sites of primers in the process of identification. (a) is a flow chart of disruption of the first CAT gene; and (b) is a flow chart of disruption of the second CAT gene.

FIG. 3 is an illustration of the means of counting the base pairs within the sequence in relation to the start codon.

FIG. 4 is the partial sequence (−423 to +420 bp) of URA3 of C. tropicalis ATTC 20336 annotated with the specific base pairs used for obtaining the gda sequences according to any aspect of the present invention.

FIG. 5 is a photo of a gel with identification results of disrupting the first CAT allele in C. tropicalis XZX by transformation of the gene disruption cassette CAT1-gda488-URA3-CAT1. Lanes 1-24 are the PCR identification results for the various transformants of the disruption cassette, and the PCR primers were CATU and CATR. Lanes 1, 7, 8, 11, 12 and 17 show positive transformants, with a single copy CAT gene being disrupted, and all of the other lanes are false positive transformants. The 2707 bp band is a band showing integration of the disruption cassette (CAT1-gda488-URA3-CAT1 fragment), and the 1881 bp band shows the CAT1 original gene.

FIG. 6 is a photo of a gel with identification results of popping-out URA3 marker gene after the first CAT allele was disrupted in C. tropicalis XZX by transformation of the disruption cassettes CAT1-gda488-URA3-CAT1 and CAT1-gda324-URA3-CAT1. Lanes 1-8 on the left side of the marker show the identification of poping-out URA3 by gda324 disruption cassette, and all lanes show strains with marker gene popped-out. The original band (sequence with CAT1 gene and a 145 bp DNA exterior of downstream homology arm) has a size of 2026 bp, and the band after marker gene pop-out (sequence of CAT1-gda324-CAT1 and a 145 bp DNA exterior of downstream homology arm) has a size of 1110 bp. DNA sequencing revealed that the sequence structure conforms with the theoretical prediction and the marker gene fragment between the two gda sequences with the same direction were popped out. Lanes 1-6 on the right side of the marker show the identification of popping-out URA3 by gda488 disruption cassette. The original band has a size of 2026 bp and the band after popping-out of marker gene (sequence of CAT1-gda488-URA3-CAT1 and a 145 bp DNA exterior of downstream homology arm) has a size of 1274 bp. Lane XZX shows PCR products using C. tropicalis XZX chromosomal DNA as a template, with a size of 2026 bp. The PCR primers were CATU/CATLD.

FIG. 7 is a photo of a gel with identification results of disrupting the first CAT allele in C. tropicalis XZX by transformation of the gene disruption cassettes CAT1-gda324-URA3-CAT1 and CAT1-gda245-URA3-CAT1. Lanes 1-12 on the left side of the marker show PCR identification results for the various transformants of the disruption cassette CAT1-gda245-URA3-CAT1 (with a size of 2464 bp). Lanes 1-5, 7-9 and 11 are positive transformants, with a PCR product (URA3-CAT1) size of 1931 bp. The other lanes are all false-positive transformants which do not have specific bands. Lanes 1-11 on the right side of the marker show PCR identification results for the various transformants of the disruption cassette CAT1-gda324-URA3-CAT1. Lanes 1-3 and 8 are positive transformants, with a PCR product (URA3-CAT1) size of 1931 bp, and the other lanes are all false-positive transformants which do not have specific bands. Lane XZX shows PCR amplification results using chromosomal DNA of C. tropicalis XZX as a template. The PCR primers were URAU/CATR.

FIG. 8 is a photo of a gel with identification results of popping-out URA3 marker gene after the first CAT allele was disrupted in C. tropicalis XZX by transformation of the disruption cassettes CAT1-gda245-URA3-CAT1 and CAT1-gda143-URA3-CAT1. Lanes 1-4 on the left side of the marker show the identification of popping-out URA3 by gda143 cassette. Lanes 1-3 are positive transformants, with an original band (a sequence of CAT1 gene and downstream sequence of the gene) size of 2026 bp and a band size after marker gene popped-out (a sequence of CAT1-gda143-CAT1 and a 145 bp DNA exterior of downstream homology arm) of 929 bp. Lanes 1-2 on the right side of the marker show the identification of popping-out URA3 by gda245 cassette, and lanes 1-2 are all positive transformants. The original band has a size of 2026 bp and the band after popping-out of marker gene (CAT1-gda245-CAT1 and a 145 bp DNA exterior of downstream homology arm) has a size of 1031 bp. The electrophoresis results of PCR products conform to the theoretically predicted size of the band after popping-out of marker gene. Lane XZX shows PCR products using chromosomal DNA of C. tropicalis XZX as a template, with a size of 2026 bp. The PCR primers were CATU/CATLD.

FIG. 9 is a photo of a gel with identification results of disrupting the first CAT allele in C. tropicalis XZX by transformation of the gene disruption cassette CAT1-gda143-URA3-CAT1. Lanes 5, 9-14, 16, 20, 23, and 24 are positive transformants, and the other lanes are all false-positive transformants. The PCR product of positive transformant (CAT1-gda143-URA3-CAT1) has a band size of 2389 bp and the original band (CAT1 original gene) has a size of 1881 bp. The primers were CATU/CATR. The band of false-positive transformants (CAT1 gene) has a size of 1881 bp.

FIG. 10 is a photo of a gel with identification results of disrupting the first CAT allele in C. tropicalis XZX by transformation of the gene disruption cassettes CAT1-gda325-URA3-CAT1 and CAT1-URA3-gda305-CAT1. Lanes 1-11 on the left side of the marker show the PCR identification results of various transformants of the disruption cassette CAT1-URA3-gda305-CAT1. Lanes 1, 4, 5, and 10 are positive transformants and the other lanes are all false-positive transformants. The band of false-positive transformants (CAT1 gene) has a size of 1881 bp, and the band of a transformant integrated by a gene disruption cassette (CAT1-URA3-gda305-CAT1) has a size of 2524 bp. Lanes 1-12 on the right side of the marker show PCR identification results of various transformants of the disruption cassette CAT1-gda325-URA3-CAT1. Lanes 3, 5, and 10-12 are positive transformants and the other lanes are all false-positive transformants. The band of PCR products of false-positive transformants (CAT1 original gene) has a size of 1881 bp and the band of a transformant integrated by a gene disruption cassette (CAT1-gda325-URA3-CAT1) has a size of 2544 bp. Lane XZX shows the result of PCR amplification using C. tropicalis XZX chromosomal DNA as a template, with a size of 1881 bp (CAT1 gene). The PCR primers were CATU/CATR.

FIG. 11 is a photo of a gel with identification results of popping-out URA3 marker gene after the first CAT allele was disrupted in C. tropicalis XZX by transformation of the disruption cassettes CAT1-gda325-URA3-CAT1 and CAT1-URA3-gda305-CAT1. Lanes 1-12 on the left side of the marker show the identification of popping-out URA3 with gda305 cassette. Lanes 1-12 are all transformants with marker gene popped-out. The original band (CAT1 gene) has a size of 1881 bp and the band after marker gene popped-out (CAT1-gda305-CAT1) has a size of 993 bp. Lanes 1-11 on the right side of the marker show the identification of popping-out URA3 by gda325 cassette. Lanes 2-10 are positive transformants. The original band (CAT1 gene) has a size of 1881 bp, and the band after popping-out of marker gene (CAT1-gda325-+858 bp to 1158 bp fragment in URA3 gene-CAT1) has a size of 1335 bp. PCR identification conformed that the band after popping-out of marker gene conformed with the theoretical prediction in size. Lane XZX is PCR product with C. tropicalis XZX chromosomal DNA as a template, with a size of 1881 bp (CAT1 gene). The PCR primers were CATU/CATR.

FIG. 12 is a photo of a gel with identification results of disrupting the first CAT allele in C. tropicalis XZX by transformation of the gene disruption cassette CAT1-URA3-gda302-CAT1. Lanes 1-7 show PCR identification results for various transformants of disruption cassette CAT1-URA3-gda302-CAT1, with lanes 5 and 7 being positive transformants and the other lanes being false-positive transformants. The band of false-positive transformants (CAT1 original gene) has a size of 1881 bp, and the positive transformants or the disruption cassette integrated transformants (CAT1-URA3-gda302-CAT1) has a band size of 2521 bp. The PCR primers were CATU/CATR.

FIG. 13 is a photo of a gel with identification results of popping-out URA3 marker gene after the first CAT allele was disrupted in C. tropicalis XZX by transformation of the gene disruption cassette CAT1-URA3-gda302-CAT1. Lanes 1-12 show identification of popping-out URA3 by gda302 cassette. All lanes show strains with successful popping out of marker gene. The original band (a sequence of CAT1 gene and a downstream sequence) has a size of 2026 bp. The band after popping-out of marker gene (CAT1-−423 bp to +16 bp in URA3 gene-gda302-CAT1 and a downstream sequence) has a size of 1425 bp. Lane XZX shows PCR products with C. tropicalis XZX chromosomal DNA as a template, with a size of 2026 bp (CAT1 gene and a downstream sequence). The PCR primers were CATU/CATLD.

FIG. 14 is a photo of a gel with identification results of disrupting the first CAT allele disrupted in C. tropicalis XZX by transformation of the gene disruption cassette CAT1-hisG-URA3-hisG-CAT1. Lanes 1-48 show PCR identification results of various transformants. Lanes 2 and 42 are positive transformants, and the other lanes are all false-positive transformants which do not have a specific amplification band. The PCR amplification band of positive transformants or the gene disruption cassette integrated transformants(hisG1) has a size of 1149 bp. The PCR primers were His-F1 and His-R1.

FIG. 15 is a photo of a gel with identification results of popping-out URA3 marker gene after the first CAT allele was disrupted in C. tropicalis XZX by transformation of the gene disruption cassette CAT1-hisG-URA3-hisG-CAT1. Lanes 1-12 show the identification of the efficiency of hisG repeat sequence to pop-out URA3 marker gene. All lanes shows strains popped-out of marker gene. The original band has a size of 2026 bp and the band after popped-out of marker gene (a sequence of CAT1-hisG-CAT1 and one CAT1 gene downstream sequence) has a size of 2078 bp. Lane XZX shows PCR product using C. tropicalis XZX chromosomal DNA as a template, with a size of 2026 bp (CAT1 gene and one downstream sequence). The PCR primers were CATU/CATLD.

FIG. 16 is a photo of a gel with identification results of disrupting the second CAT allele in C. tropicalis 02 (URA3/URA3, cat::gda324/CAT) by transformation of the gene disruption cassette CAT2-gda324-URA3-CAT2. Lanes 1-12 show PCR identification results for various transformants. Lanes 1, 3, 5, 8, and 9 are positive transformants, and the other lanes are all false-positive transformants. The false-positive transformant does not have a specific amplification band. The disruption cassette integrated transformant had a PCR amplification band (CAT2-gda324-URA3-CAT2-fragment from downstream homology arm of CAT2 to downstream homology arm of CAT1-CAT1) size of 3027 bp. Lane XZX shows PCR products with C. tropicalis XZX chromosomal DNA as a template (CAT gene fragment from CAT2 gene upstream homology arm to CAT1 downstream homology arm), which has size of 1312 bp. The PCR primers were CAT2ndU/CATR.

FIG. 17 is a photo of a gel with identification results of popping-out URA3 marker gene after the second CAT allele was disrupted in C. tropicalis 02 by transformation of the gene disruption cassette CAT2-gda324-URA3-CAT2. Lanes 1-3 show the identification of popping-out of URA3 by gda324 cassette. All lanes show strains with marker gene popped-out. The band with popped-out marker gene (CAT2-gda324-CAT2-fragment between CAT2 downstream homology arm and CAT1 downstream homology arm-CAT1) has a size of 1444 bp. Lane XZX shows PCR products with the C. tropicalis XZX chromosome as a template, with an original band (a CAT1 gene fragment between CAT2 upstream homology arm to CAT1 downstream homology arm) size of 1312 bp. The PCR primers were CAT2ndU/CATR.

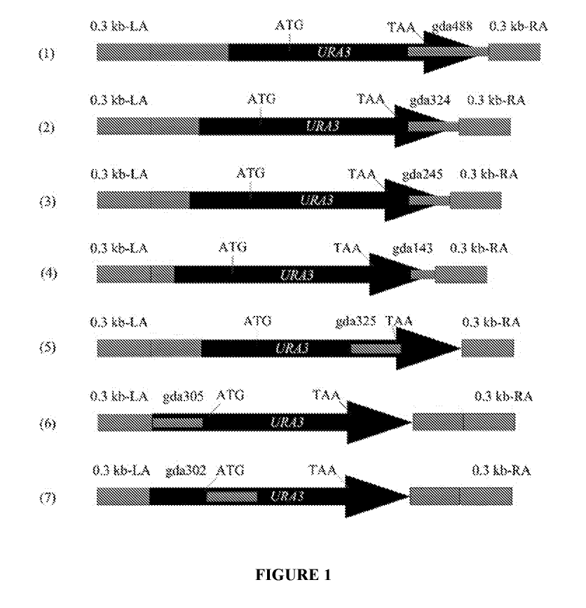

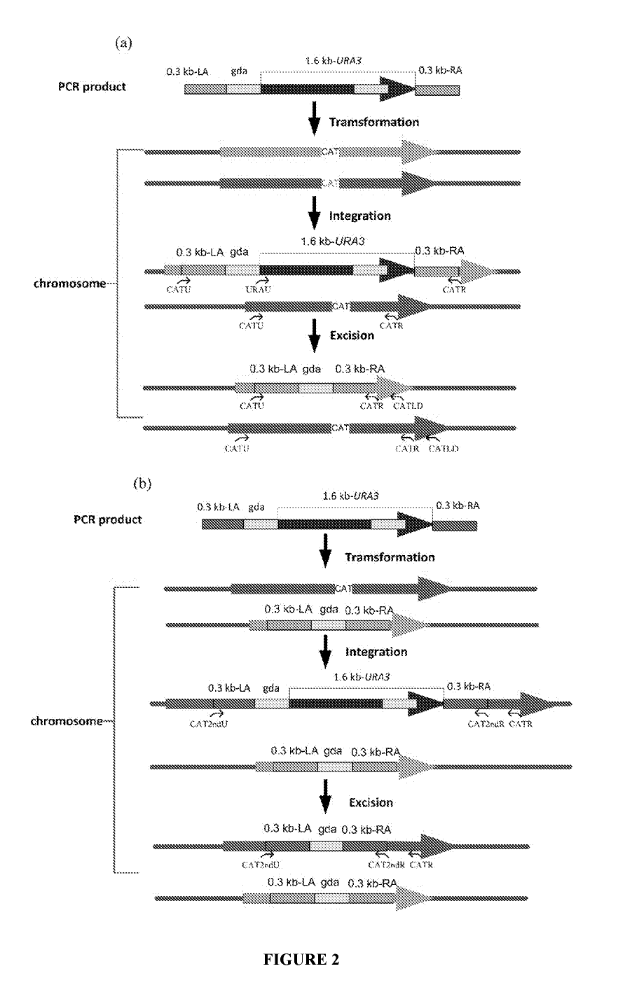

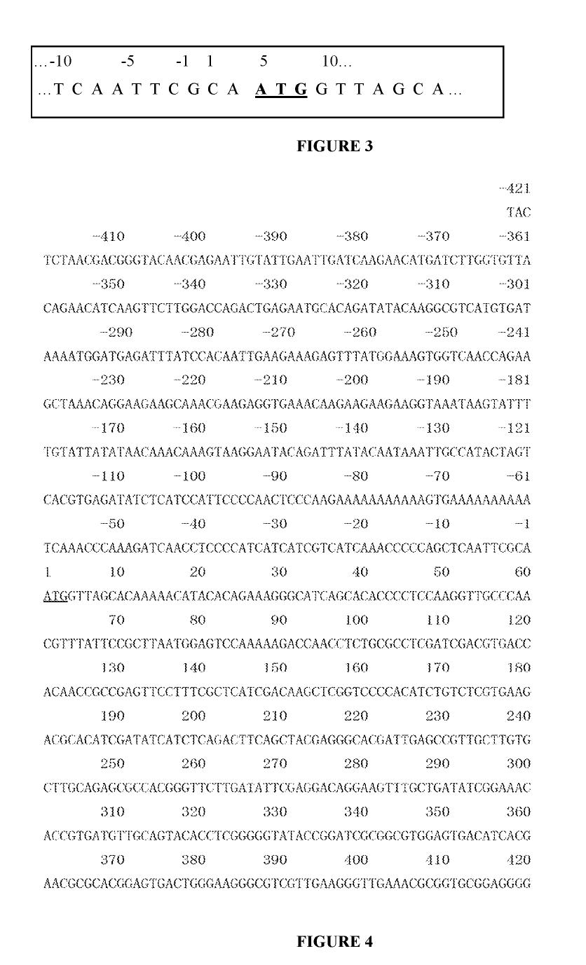

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Methods and Materials

Uracil auxotroph strain C. tropicalis XZX was used as a target strain for gene disruption. The uracil auxotroph strain was derived by screening of C. tropicalis ATTC 20336 after physical or chemical mutagenesis, and the open reading frame of the URA3 gene of the mutant strain comprised a missense mutation which altered the amino acid sequence.

The specific method is as follows: C. tropicalis ATTC 20336 as the starting strain was subjected to mutagenesis 11 times and screened with FOA selection medium, and a total of 127 colonies grew from the FOA selection medium (SM+5-fluoroorotic acid 2 g/L). The grown colonies were separately cultured on a SM plate and a MM plate. Finally, 13 URA3/URA3 mutant strains were identified; 3 of the 13 strains were selected, and designated as C. tropicalis XZW, C. tropicalis XZX, and C. tropicalis XZB respectively. DNA sequencing analysis showed that the common mutation in the URA3 gene sequence was the mutation of base G to A happening at the base pair at position +608. This mutation in base, which incurred, changed the protein sequence, and was the main cause of the functional defect of the URA3 gene (see Zheng Xiang, Xianzhong Chen et al. 2014).

The following culture media and compositions were used in the examples of the present invention: MM (yeast nitrogen base without amino acids & ammonium sulfate, YNB 6.7 g/L; glucose 20 g/L; (NH4)2SO4 10 g/L); SM (MM+uracil 60 mg/L); and FOA culture medium (SM+5-fluoroorotic acid 2 g/L).

The recombination efficiency calculated in all the examples and the results shown in table 1 was calculated according to the following formula:

$$Recombination efficiency = \frac{\frac{\begin{matrix}\begin{matrix}Total no . of transformants \times \\ no . of transformants dentified as\end{matrix} \\ correct transformation\end{matrix}}{no . of transformants i dentified}}{total DNA weight ( μg )}$$

The marker gene pop-out efficiency calculated in all the examples and the results shown in the table 2 was calculated according to the following formula:

$$Pop \text{-} out efficiency = \frac{\frac{\begin{matrix}\begin{matrix}\begin{matrix}average no . of the total \\ colonies on 3 FOA plates \times \end{matrix} \\ no . of transformants i dentified as\end{matrix} \\ marker gene popped \text{-}\end{matrix}}{no . of identified transformants}}{total no . of cells applied to FOA plate}$$

### Example 1

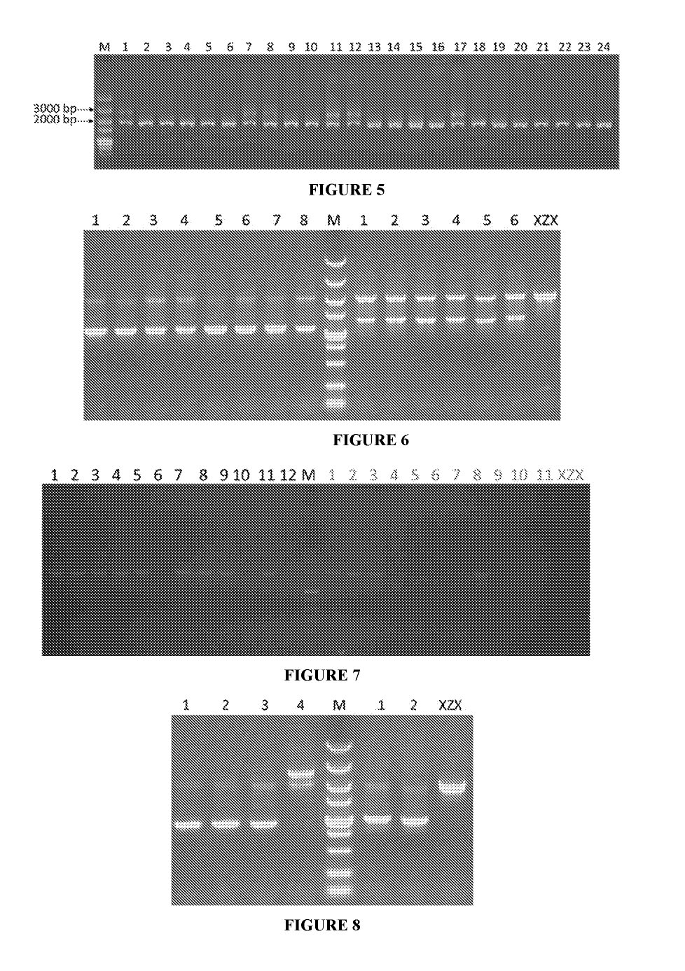

Disruption of First CAT Gene in C. tropicalis XZX by Transformation of the Gene Disruption Cassette CAT1-Gda488-URA3-CAT1
1. Culturing of C. tropicalis ATTC 20336 strain.
   C. tropicalis ATTC 20336 was inoculated in SM or MM medium, and cultured in a shake flask at 30° C., 200 rpm until the desired microbial concentration was reached to extract chromosomal DNA.
2. Isolation of C. tropicalis ATTC 20336 chromosomal DNA.
   (1) Centrifugation was carried out to obtain the cells; (2) a suitable amount of sorbitol-Na₂EDTA buffer solution (sorbitol 1 mol/L, Na₂EDTA 0.1 mol/L, pH 7.5) was added to form a microbial suspension, a suitable amount of Snailase solution (50 mg/mL) was next added, and after mixing until uniform, digestion was carried out at 37° C. for 4 h in order to remove the yeast cell walls; (3) centrifugation was carried out to collect the cells, the supernatant was discarded, a suitable amount of Tris-HCl-Na₂EDTA solution (Tris 50 mmol/L, Na₂EDTA 20 mmol/L, pH 7.4) was used to gently suspend the cells, a suitable amount of SDS solution (SDS 100 g/L) was added, and the mixture was stirred until uniform and incubated at 65° C. for 30 min; (4) after the microbial suspension became clear, 200 μL of potassium acetate solution (potassium acetate 5 mol/L) was added, the mixture was stirred until uniform, and it was placed for 1 h in an ice bath; (5) centrifugation was carried out at 12000 rpm for 5 min. The supernatant was transferred to a fresh EP tube, an equivalent volume of isopropanol was added, and the mixture was stirred until uniform and then allowed to stand at room temperature for 15 min; (6) centrifugation was carried out at 12000 rpm for 5 min, the supernatant was discarded, the precipitation were washed with 200 μL of 70% ethanol solution. The ethanol solution was discarded, the precipitate was allowed to dry naturally, 33 μL of sterile water was added to dissolve the precipitation, 2 μL of RNaseA was added, the mixture was stirred until uniform, and it was incubated at 37° C. for 1 h to digest the RNA; (7) after incubation was completed, the C. tropicalis ATTC 20336 chromosomal DNA was obtained, which could be applied directly as a PCR template or stored at −20° C.
3. Preparation of URA3 gene fragment and Tm-URA3 vector
   C. tropicalis ATTC 20336 chromosomal DNA was used as a template, the URA3 gene upstream primer URAU: 5′-tactctaacgacgggtacaac-3′ (SEQ ID NO: 1), and the downstream primer URAR: 5′-acccgatttcaaaagtgcaga-3′ (SEQ ID NO: 2) were designed according to the URA3 gene of C. tropicalis in NCBI (GenBank Accession No. AB006207), PCR amplification was conducted to produce a URA3 gene fragment (SEQ ID NO: 3) with a size of 1581 bp. The URA3 gene fragment was ligated to a commercial vector pMD18-T Vector (Takara Biotechnology (Dalian) Co., Ltd, Dalian, China) to obtain a recombinant plasmid, which was then introduced into E. coli JM109 for amplification. The recombinant plasmid was designated as Tm-URA3.
4. Preparation of gda488 sequence (URA3 gene fragment from +671 to +1158).
   C. tropicalis ATTC 20336 chromosomal DNA was used as a template, and the synthetic gda488 sequence formed using the upstream primer Ugda488 5′-aactgcagttctgactggtaccgat-3′ (SEQ ID NO: 4) and the downstream primer Dgda 5′-gcgtcgacacccgatttcaaaagtgcaga-3′ (SEQ ID NO: 5) were used in PCR. PCR amplification was conducted to produce a gda488 sequence (SEQ ID NO. 6).
5. PstI and SalI were used to double digest the above gda488 fragment and the recombinant vector Tm-URA3. Then ligating to form a recombinant plasmid, which was introduced into E. coli JM109 for amplification. This new recombinant plasmid was designated as Tm-gda488-URA3.
6. C. tropicalis ATTC 20336 chromosomal DNA was used as a template, CAT gene upstream primer CATU 5′-gtttaactttaagttgtcgc-3′ (SEQ ID NO: 7), and the downstream primer CATR: 5′-tacaacttaggcttagcatca-3′ (SEQ ID NO: 8) were used in PCR. PCR amplification was conducted to produce a CAT1 gene (SEQ ID NO: 9) with a size of 1881 bp, after which it was ligated to a pMD18-T Simple Vector (Takara Biotechnology (Dalian) Co., Ltd, Dalian, China) commercial vector to obtain a recombinant plasmid, which was introduced into E. coli JM109 for amplification. The recombinant plasmid was designated as Ts-CAT1.
7. Recombinant plasmid Ts-CAT1 was used as a template, inverse PCR primer rCATU: 5′-aactgcagccaaaattcagccaaccagt-3′ (SEQ ID NO: 10), and rCATR: 5′-gctctagaagatgattcaaccaggcgaac-3′ (SEQ ID NO: 11) were used to amplify by inverse PCR and to obtain a fragment with upstream and downstream CAT gene homology arms, which was designated as CAT1-Ts-CAT1.
8. Restriction endonuclease PstI and XbaI were used to double digest vector Tm-gda488-URA3. The gda488-URA3 fragment was recovered and ligated with a PstI and XbaI double digested CAT1-Ts-CAT1 fragment to form a recombinant plasmid. Then the plasmid was introduced into E. coli JM109 for amplification. The recombinant plasmid was designated as Ts-CAT1-gda488-URA3.
9. Using recombinant plasmid Ts-CAT1-gda488-URA3 as a template and CAT gene up/downstream primer CATU/CATR, PCR amplification was carried out to obtain a first CAT allele gene disruption cassette, designated as CAT1-gda488-URA3-CAT1.
10. The fully-constructed gene disruption cassette CAT1-gda488-URA3-CAT1 was transformed using the lithium chloride transformation method into uracil auxotroph C. tropicalis XZX and then applied onto a MM plate. After growth of the transformants was completed, chromosomal DNA isolated according to method of steps 1 and 2 was used in PCR identification, and the strain that was identified as correct transformants was designated as strain 01-1. The PCR identification primers were CATU and CATR. The total number of transformants on MM plate was 28, and the number of transformants identified was 24, the number of transformants identified as correct transformation was 6, the recombination efficiency was 1 transformant/ng DNA (Table 1). PCR identification results are shown in FIG. 5.
11. A single colony of strain 01-1 was inoculated into a SM liquid medium, cultured in shake flask at 30° C. and 200 rpm until a specified cell concentration of OD₆₀₀ of 13 to 15 was reached. The cells were then diluted and applied to an SM plate for statistical determination of cell concentration; it was simultaneously applied onto an FOA plate and cultured at 30° C.
12. After 3 days, the SM plate count was calculated; after 5 days, the number of mutant strains on the FOA plate was calculated, and the single colonies were picked and inoculated in SM culture.
13. Chromosomal DNA isolated according to method of steps 1 and 2 was identified by PCR. The PCR identification primers were CATU and primer CATLD 5′-aatagaaactagcaatcggaa-3′ (SEQ ID NO: 12) from the outer side of CAT gene downstream sequence. The strains showing successful URA3 marker gene loss were identified was designated as strain 02. PCR was used to identify the successful strains which had expression of the popping-out URA3 marker gene after the first CAT allele was disrupted in C. tropicalis XZX by transformation of the disruption cassettes CAT1-gda488-URA3-CAT1 and CAT1-gda324-URA3-CAT1. DNA sequencing revealed that the sequence structure of the PCR product CAT1-gda324-CAT1-CATLD at the CAT gene locus after the marker gene was popped out conforms to the theoretical prediction (identity of the two sequences was 97.23%) and the marker gene fragment between the two gda sequences with the same direction were popped out. The results also showed that the identification of popping-out URA3 by gda488 disruption cassette. The original band has a size of 2026 bp and the band after popping-out of marker gene (sequence of CAT1-gda488-URA3-CAT1 and a 145 bp DNA exterior of downstream homology arm) has a size of 1274 bp. Statistical results for marker gene pop-out efficiency are shown in Table 2. PCR identification results are shown in FIG. 6.

### Example 2

Disruption of First CAT Gene in C. tropicalis XZX by Transformation of the Gene Disruption Cassette CAT1-Gda324-URA3-CAT1
1. Using C. tropicalis ATTC 20336 chromosomal DNA as a template, the synthetic gda324 sequence upstream primer Ugda324 5′-aactgcagactaagcttctaggacgtcat-3′ (SEQ ID NO. 13), and the downstream primer Dgda(SEQ ID NO:5) as primers, PCR amplification was conducted to produce a gda324 sequence (URA3 gene fragment from +835 to +1158) (SEQ ID NO. 14).
2. PstI and SalI were used to double digest the above gda324 fragment and the recombinant vector Tm-URA3, the fragments were ligated to form a new recombinant plasmid, which was introduced into E. coli JM109 for amplification. This plasmid was designated as Tm-gda324-URA3.
3. PstI and XbaI were used to double digest vector Tm-gda324-URA3. The gda324-URA3 fragment was recovered, and ligated to the PstI and XbaI double digested CAT1-Ts-CAT1 fragment to form a recombinant plasmid, which was introduced into E. coli JM109 for amplification. This plasmid was designated as Ts-CAT1-gda324-URA3.
4. Using recombinant plasmid Ts-CAT1-gda324-URA3 as a template, PCR amplification was carried out according to the method of step 9 of Example 1 to obtain a first CAT allele disruption cassette CAT1-gda324-URA3-CAT1.
5. The XZX strain was transformed according to step 10 of Example 1 and PCR identification was carried out. PCR identification results are shown in FIG. 7. The total number of transformants on MM plate was 17, the number of transformants identified was 11, the number of transformants identified as correctly transformed was 4, the recombination efficiency was 1.24 transformants/μg DNA (see Table 1). The results specifically showed the various transformants of the disruption cassette CAT1-gda245-URA3-CAT1 (with a size of 2464 bp), the positive transformants, with a PCR product (URA3-CAT1) size of 1931 bp, false-positive transformants which did not have specific bands and various transformants of the disruption cassette CAT1-gda324-URA3-CAT1. The PCR results also showed positive transformants, with a PCR product (URA3-CAT1) size of 1931 bp. Chromosomal DNA of C. tropicalis XZX as used as a template and control. The PCR primers were URAU/CATR.
6. Marker gene loss was carried out according to steps 11-13 of Example 1, with PCR identification. The results are shown in FIG. 6. Lanes 1-8 on the left side of the marker are strains with popped-out marker gene. Statistical results for marker gene pop-out efficiency are shown in Table 2.

### Example 3

Disruption of the First CAT Gene in C. tropicalis XZX by Transformation of the Gene Disruption Cassette CAT1-Gda245-URA3-CAT1
1. Using C. tropicalis ATTC 20336 chromosomal DNA as a template, the synthetic gda245 sequence upstream primer Ugda 5′-aactgcagaatggatgtagcagggatggt-3′ (SEQ ID NO: 15) and the downstream primer Dgda(SEQ ID NO:5) as primers, PCR amplification was conducted to produce a gda245 sequence (URA3 gene fragment from +914 to +1158) (SEQ ID NO: 16).
2. PstI and SalI were used in a double digest the above gda245 fragment and the recombinant vector Tm-URA3. The fragments were ligated to form a recombinant plasmid, which was introduced into E. coli JM109 for amplification. This plasmid was designated as Tm-gda245-URA3.
3. Vector Tm-gda245-URA3 was double digested with PstI and XbaI. The gda245-URA3 fragment was recovered and ligated to the PstI and XbaI double digested CAT1-Ts-CAT1 fragment to form a recombinant plasmid, which was introduced into E. coli JM109 for amplification. This plasmid was designated as Ts-CAT1-gda245-URA3.
4. Using recombinant plasmid Ts-CAT1-gda245-URA3 as a template, PCR amplification was carried out according to the method of step 9 of Example 1 to obtain a first CAT allele disruption cassette CAT1-gda245-URA3-CAT1.
5. The XZX strain was transformed according to the method of step 10 of Example 1 and PCR identification was carried out with the primers URAU/CATR. The identification results as shown in FIG. 7, showed that the total number of transformants on MM plate was 21, number of transformants identified was 12, number of transformants identified as correctly transformed was 9, and the recombination efficiency was 2.24 transformants/μg DNA (see Table 1).
6. Marker gene loss was carried out according to steps 11-13 of Example 1, and PCR identification results are shown in FIG. 8 of popping-out URA3 marker gene after the first CAT allele was disrupted in C. tropicalis XZX by transformation of the disruption cassette CAT1-gda245-URA3-CAT1 showed that the PCR products conform with the theoretically predicted size of the band after popping-out of marker gene. Statistical results for marker gene pop-out efficiency are shown in Table 2.

### Example 4

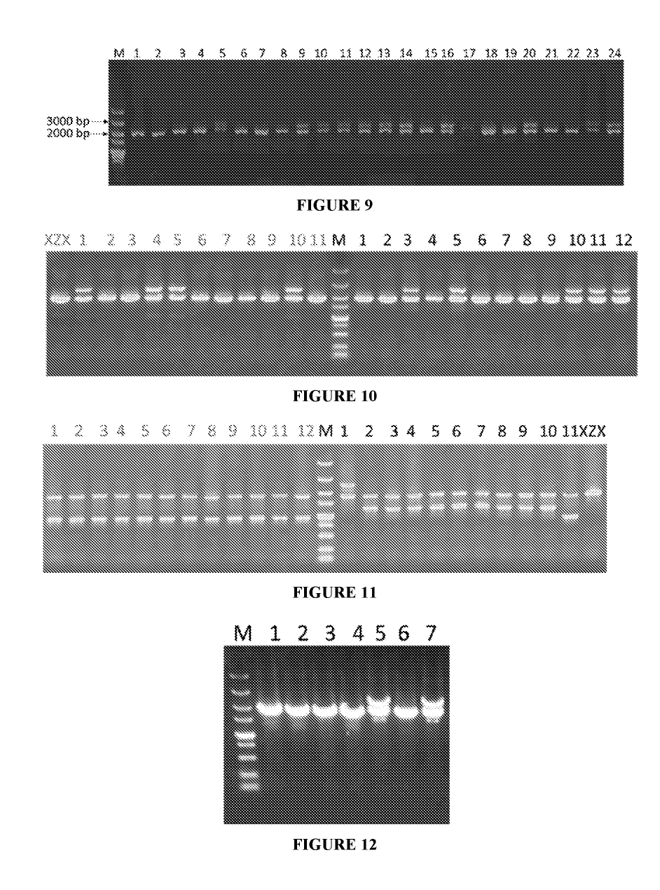

Disruption of the First CAT Gene in C. tropicalis XZX by Transformation of the Gene Disruption Cassette CAT1-Gda143-URA3-CAT1
1. Using C. tropicalis ATTC 20336 chromosomal DNA as a template, the synthetic gda143 sequence upstream primer Ugda143 5′-aactgcagtgcttgaaggtattcacgta-3′ (SEQ ID NO: 17), and the downstream primer Dgda(SEQ ID NO: 5), as primers, PCR amplification was conducted to produce a gda143 sequence (URA3 gene fragment from +1016 to +1158) (SEQ ID NO. 18).
2. PstI and SalI were used to double digest the above gda143 fragment and the recombinant vector Tm-URA3. The fragments were ligated to form a recombinant plasmid, which was introduced into E. coli JM109 for amplification. This plasmid was designated as Tm-gda143-URA3.
3. Vector Tm-gda143-URA3 was double digested by PstI and XbaI. The gda143-URA3 fragment was recovered and ligated to the PstI and XbaI double digested CAT1-Ts-CAT1 fragment to form a recombinant plasmid, which was introduced into E. coli JM109 for amplification. This plasmid was designated as Ts-CAT1-gda143-URA3.
4. Using recombinant plasmid Ts-CAT1-gda143-URA3 as a template, PCR amplification was carried out according to the method of step 9 of Example 1 to obtain a first CAT allele disruption cassette CAT1-gda143-URA3-CAT1.
5. The XZX strain was transformed according to the method of step 10 of Example 1 and PCR identification was carried out and the results showed that the total number of transformants on MM plate was 31, number of transformants identified was 24, number of transformants identified as correctly transformed was 11, and the recombination efficiency was 1.95 transformants/μg DNA (see Table 1). PCR identification results are shown in FIG. 9.
6. Marker gene loss was carried out according to the method of steps 11-13 of Example 1, and the PCR identification results of popping-out URA3 marker gene after the first CAT allele was disrupted in C. tropicalis XZX by transformation of the disruption cassette CAT1-gda143-URA3-CAT1 showed that the PCR products conform with the theoretically predicted size of the band after popping-out of marker gene. Statistical results for marker gene pop-out efficiency are shown in Table 2. PCR identification results are shown in FIG. 8.

### Example 5

Disruption of the first CAT gene in C. tropicalis XZX by transformation of the gene disruption cassette CAT1-gda325-URA3-CAT1 1. Using C. tropicalis ATTC 20336 chromosomal DNA as a template, the synthetic gda325 sequence upstream primer Ugda325 5′-aactgcagtcgtgattgggttcatcgc-3′ (SEQ ID NO. 19), and the downstream primer Dgda325 5′-gcgtcgaccaatgacgtcctagaagc-3′ (SEQ ID NO. 20) as primers, PCR amplification was conducted to produce a gda325 sequence (URA3 gene fragment from +533 to +857) (SEQ ID NO. 21).
2. PstI and SalI were used to double digest the above gda325 fragment and the recombinant vector Tm-URA3. The fragments were ligated to form a recombinant plasmid, which was introduced into E. coli JM109 for amplification. This plasmid was designated as Tm-gda325-URA3.
3. Vector Tm-gda325-URA3 was double digested with PstI and XbaI. The gda325-URA3 fragment was recovered and ligated to the PstI and XbaI double digested CAT1-Ts-CAT1 fragment to form a recombinant plasmid, which was introduced into E. coli JM109 for amplification. This plasmid was designated as Ts-CAT1-gda325-URA3.
4. Using recombinant plasmid Ts-CAT1-gda325-URA3 as a template, PCR amplification was carried out according to the method of step 9 of Example 1 to obtain a first CAT allele disruption cassette CAT1-gda325-URA3-CAT1.
5. The XZX strain was transformed according to the method of step 10 of Example 1 and PCR identification was carried out (results shown in FIG. 10), with the primers URAU/CATR.
6. Marker gene loss was carried out according to the method of steps 11-13 of Example 1, and PCR identification results showed identification results of popping-out URA3 marker gene after the first CAT allele was disrupted in C. tropicalis XZX by transformation of the disruption cassette CAT1-gda325-URA3-CAT1. In particular, the results showed that the identification of popping-out URA3 by gda325 cassette. The original band (CAT1 gene) had a size of 1881 bp, and the band after popping-out of marker gene (CAT1-gda325-+858 bp to 1158 bp fragment in URA3 gene-CAT1) had a size of 1335 bp. PCR identification (results shown in FIG. 11) confirmed that the band after popping-out of marker gene conformed with the theoretical prediction in size. The control was a PCR product with C. tropicalis XZX chromosomal DNA as a template, with a size of 1881 bp (CAT1 gene). The PCR identification primers were CATU/CATR. Statistical results for marker gene pop-out efficiency are shown in Table 2.

### Example 6

Disruption of the First CAT Gene in C. tropicalis XZX by Transformation of the Gene Disruption Cassette CAT1-URA3-Gda305-CAT1
1. Using C. tropicalis ATTC 20336 chromosomal DNA as a template, the synthetic gda305 sequence upstream primer Ugda305 5′-gctctagatctaacgacgggtacaacga-3′ (SEQ ID NO: 22), and the downstream primer Dgda305 5′-cggaattcacgtgactagtatggcaat-3′ (SEQ ID NO: 23) as primers, PCR amplification was conducted to produce a gda305 sequence (URA3 gene fragment from −420 to −116) (SEQ ID NO: 24).
2. XbaI and EcoRI were used to double digest the above gda305 fragment and the recombinant vector Tm-URA3. The fragments were ligated to form a recombinant plasmid, which was introduced into E. coli JM109 for amplification. This plasmid was designated as Tm-URA3-gda305.
3. Vector Tm-URA3-gda305 was double digested by PstI and EcoRI. The URA3-gda305 fragment was recovered. PstI and XbaI were used to double digest CAT1-Ts-CAT1. pfu DNA polymerase was then used to fill in the sticky ends of the CAT1-Ts-CAT1 and dp1305-URA3 fragments in order to carry out blunt end ligation and obtain a recombinant plasmid, which was then introduced into E. coli JM109 for amplification. This plasmid was designated as Ts-CAT1-URA3-gda305.
4. Using recombinant plasmid Ts-CAT1-URA3-gda305 as a template, PCR amplification was carried out according to the method of step 9 of Example 1 to obtain a first CAT allele disruption cassette CAT1-URA3-gda305-CAT1.
5. The XZX strain was transformed according to the method of step 10 of Example 1 and PCR identification was carried out, and the identification results showed the disruption of the first CAT allele in C. tropicalis XZX by transformation of the gene disruption cassettes CAT1-gda325-URA3-CAT1 (Example 5) and CAT1-URA3-gda305-CAT1. The successful transformants (true positives) were selected for the next step. PCR identification results are shown in FIG. 10.
6. Marker gene loss was carried out according to the method of steps 11-13 of Example 1, and PCR identification results showed identification results of popping-out URA3 marker gene after the first CAT allele was disrupted in C. tropicalis XZX by transformation of the disruption cassette CAT1-gda305-URA3-CAT1. In particular, the results showed that the identification of popping-out URA3 by gda305 cassette. The original band (CAT1 gene) had a size of 1881 bp, and the band after marker gene popped-out (CAT1-gda305-CAT1) had a size of 993 bp. PCR identification confirmed that the band after popping-out of marker gene conformed to the theoretical prediction in size. The control was a PCR product with C. tropicalis XZX chromosomal DNA as a template, with a size of 1881 bp (CAT1 gene). The PCR identification primers were CAT1/CATR. Statistical results for marker gene pop-out efficiency are shown in Table 2. PCR identification results are shown in FIG. 11.

### Example 7

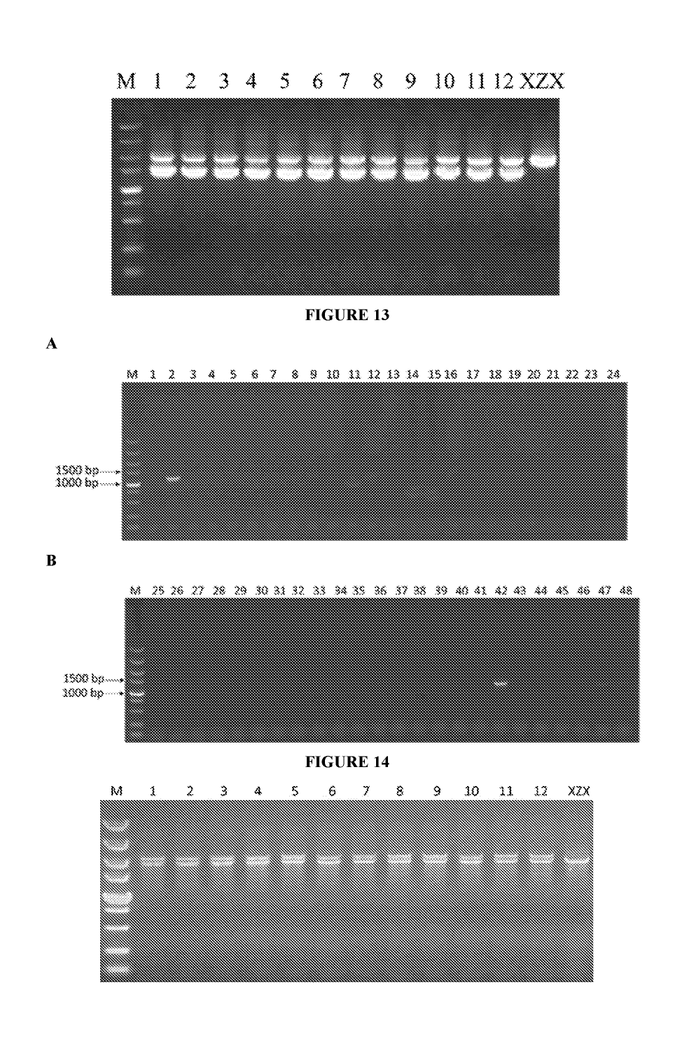

Disruption of the First CAT Gene in C. tropicalis XZX by Transformation of the Gene Disruption Cassette CAT1-URA3-Gda302-CAT1
1. Using C. tropicalis ATTC 20336 chromosomal DNA as a template, the synthetic gda302 sequence upstream primer Ugda302 5′-gctctagacatacacagaaagggcatc-3′ (SEQ ID NO: 25), and the downstream primer Dgda302 5′-cggaattcgtactgcaacatcacgg-3′ (SEQ ID NO: 26) as primers, PCR amplification was conducted to produce a gda302 sequence (URA3 gene fragment from +17 to +318) (SEQ ID NO: 27).
2. XbaI and EcoRI were used to double digest the above gda302 fragment and the recombinant vector Tm-URA3. The fragments were ligated to form a recombinant plasmid, which was introduced into E. coli JM109 for amplification. This plasmid was designated as Tm-URA3-gda302.
3. Vector Tm-URA3-gda302 were double digested with PstI and EcoRI. The URA3-gda302 fragment was recovered. PstI and XbaI were used to double digest CAT1-Ts-CAT1. pfu DNA polymerase was then used to fill in the sticky ends of the CAT1-Ts-CAT1 and URA3-gda302 fragments in order to carry out blunt end ligation and obtain a recombinant plasmid, which was then introduced into E. coli JM109 for amplification. This plasmid was designated as Ts-CAT1-URA3-gda302.
4. Using recombinant plasmid Ts-CAT1-URA3-gda302 as a template, PCR amplification was carried out according to the method of step 9 of Example 1 to obtain a first CAT allele disruption cassette CAT1-URA3-gda302-CAT1.
5. The XZX strain was transformed according to the method of step 10 of Example 1, PCR identification was carried out, where the identification results showed the success of disrupting the first CAT allele in C. tropicalis XZX by transformation of the gene disruption cassette CAT1-URA3-gda302-CAT1. The band of false-positive transformants (CAT1 original gene) had a size of 1881 bp, and the positive transformants or the disruption cassette integrated transformants (CAT1-URA3-gda302-CAT1) had a band size of 2521 bp. The PCR primers were CATU/CATR. PCR identification results are shown in FIG. 12.
6. Marker gene loss was carried out according to the method of steps 11-13 of Example 1, and PCR identification results showed the results of popping-out URA3 marker gene after the first CAT allele was disrupted in C. tropicalis XZX by transformation of the gene disruption cassette CAT1-URA3-gda302-CAT1. All lanes showed strains with successful popping out of marker gene. The original band (a sequence of CAT1 gene and a downstream sequence) had a size of 2026 bp. The band after popping-out of marker gene (CAT1-−423 bp to +16 bp in URA3 gene-gda302-CAT1 and a downstream sequence) had a size of 1425 bp. The control used was PCR products with C. tropicalis XZX chromosomal DNA as a template, with a size of 2026 bp (CAT1 gene and a downstream sequence). The PCR primers were CATU/CATLD. Statistical results for marker gene pop-out efficiency are shown in Table 2. PCR identification results are shown in FIG. 13.

### Comparative Example 1

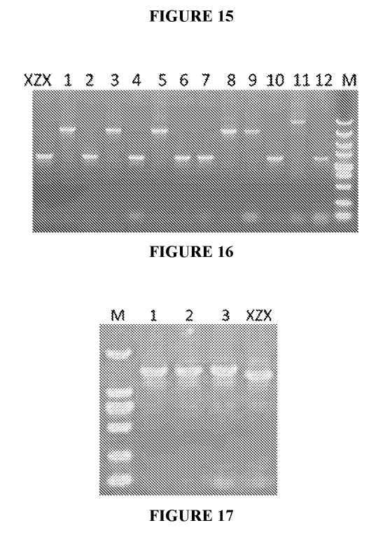

Disruption of the First CAT Gene in C. tropicalis XZX by Transformation of the Gene Disruption Cassette CAT1-hisG-URA3-hisG-CAT1
1. Isolation of hisG fragment: PCR amplification was carried out using the two pairs of primers hisG-F1 5′-ccggaattcttccagtggtgcatgaacgc-3′ (SEQ ID NO: 28) and hisG-R1 5′-cgcggattcgctgttccagtcaatcagggt-3′ (SEQ ID NO: 29) as well as hisG-F2 5′-acgcgtcgacttccagtggtgcatgaacgc-3′ (SEQ ID NO: 30) and hisG-R2 5′-aactgcaggctgttccagtcaatcagggt-3′ (SEQ ID NO: 31). PCR was carried out as taught in Ko et al. (2006), using plasmid pCUB6 as a template to obtain two 1.1 kb hisG fragments. These were designated:
   hisG1 (SEQ ID NO: 32) where the two ends had EcoRI and Bam HI restriction enzyme loci; and
   hisG2 (SEQ ID NO: 33) where the two ends had SalI and PstI restriction enzyme loci.
2. The restriction enzymes EcoRI and Bam HI were used to digest the hisG1 fragment, then the digested fragment was inserted into a Tm-URA3 plasmid that had been digested with the same enzymes to obtain the recombinant plasmid Tm-hisG1-URA3.
3. The restriction enzymes PstI and SalI were used to digest the hisG2 fragment, then the digested fragment was inserted into a Tm-hisG1-URA3 plasmid that had been digested with the same enzymes to obtain the recombinant plasmid Tm-hisG1-URA3-hisG2, abbreviated as Tm-HUH.
4. PstI and EcoRI were used to double digest the recombinant plasmid Tm-HUH, and gel recycling was used to obtain a hisG1-URA3-hisG2 fragment; PstI and XbaI were used to double digest CAT1-Ts-CAT1; pfu DNA polymerase was then used to fill in the sticky ends of the CAT1-Ts-CAT1 and hisG1-URA3-hisG2 fragment in order to carry out blunt end ligation to obtain the recombinant plasmid Ts-CAT1-hisG1-URA3-hisG2.
5. Using recombinant plasmid Ts-CAT1-hisG1-URA3-hisG2 as a template, PCR amplification was carried out according to the method of step 9 of Example 1 to obtain the first CAT allele disruption cassette CAT1-hisG1-URA3-hisG2-CAT1.
6. The XZX strain was transformed according to the method of step 10 of Example 1, PCR identification was carried out, where the identification results showed the disruption of the first CAT allele disrupted in C. tropicalis XZX by transformation of the gene disruption cassette CAT1-hisG-URA3-hisG-CAT1. The PCR amplification band of positive transformants or the gene disruption cassette integrated transformants (hisG1) had a size of 1149 bp. The PCR primers used were His-F1 and His-R1. PCR identification results are shown in FIG. 14.
7. Marker gene loss was carried out according to the method of steps 11-13 of Example 1, with PCR identification results showing the popping-out of URA3 marker gene after the first CAT allele was disrupted in C. tropicalis XZX by transformation of the gene disruption cassette CAT1-hisG-URA3-hisG-CAT1. All lanes showed strains with popped-out marker gene. The original band had a size of 2026 bp and the band after popped-out of marker gene (a sequence of CAT1-hisG-CAT1 and one CAT1 gene downstream sequence) had a size of 2078 bp. The control used C. tropicalis XZX chromosomal DNA as a template, with a size of 2026 bp (CAT1 gene and one downstream sequence). The PCR primers were CATU/CATLD. Statistical results for marker gene pop-out efficiency are shown in Table 2. PCR identification results are shown in FIG. 15.

### Example 8

Disruption of the Second CAT Allele in C. tropicalis02 (URA3/URA3, cat::gda324/CAT) by transformation of the gene disruption cassette CAT2-gda324-URA3-CAT2
1. Using C. tropicalis ATTC 20336 chromosomal DNA as a template, the CAT2 upstream primer CAT2ndU 5′-ctgaaggctccgacatcacc-3′ (SEQ ID NO; 34), and the CAT2 downstream primer CAT2ndR: 5′-caaccttgtcggcgctgcta-3′ (SEQ ID NO: 35) as primers, PCR amplification was conducted to produce a CAT2 fragment (SEQ ID NO: 36), after which it was linked to a commercial vector pMD18-T Simple Vector to obtain a recombinant plasmid, which was introduced into E. coli JM109 for amplification. The recombinant plasmid was designated as Ts-CAT2.
2. Using recombinant plasmid Ts-CAT2 as a template, the inverse PCR upstream primer rCAR2ndU: 5′-aactgcagatctgttttgaccgtccccgtg-3′ (SEQ ID NO: 37), and the downstream primer rCAT2ndR: 5′-aactgcagatctgttttgaccgtccccgtg-3′ (SEQ ID NO: 38) as primers, inverse PCR amplification was carried out to obtain a fragment having upstream and downstream CAT2 gene homology arms, which was designated as CAT2-Ts-CAT2.
3. PstI and XbaI were used to double digest vector Tm-gda324-URA3. The gda324-URA3 fragment was recovered and ligated to the PstI and XbaI double digested CAT2-Ts-CAT2 fragment to form a recombinant plasmid, which was designated as Ts-CAT2-gda324-URA3. The plasmid was introduced into E. coli JM109 for amplification.
4. Using recombinant plasmid Ts-CAT2-gda324-URA3 as a template and the CAT2 gene fragment upstream and the downstream primers CAT2ndU and CAT2ndR as primers, PCR amplification was carried out to obtain a second CAT allele disruption cassette designated as CAT2-gda324-URA3-CAT2.
5. Strain 02 from Example 1 was transformed according to the method of step 10 of Example 1, PCR identification was carried out using CAT2ndU and CATR as primers to identify the successful strains with disruption of the second CAT allele in C. tropicalis 02 (URA3/URA3, cat::gda324/CAT) by transformation of the gene disruption cassette CAT2-gda324-URA3-CAT2. The disruption cassette integrated transformant had a PCR amplification band (CAT2-gda324-URA3-CAT2-fragment from downstream homology arm of CAT2 to downstream homology arm of CAT1-CAT1) size of 3027 bp. The control used was PCR products with C. tropicalis XZX chromosomal DNA as a template (CAT gene fragment from CAT2 gene upstream homology arm to CAT1 downstream homology arm), which has a size of 1312 bp. The PCR primers were CAT2ndU/CATR. PCR identification results are shown in FIG. 16.
6. Marker gene loss was carried out according to the method of steps 11-13 of Example 1, and PCR identification was carried out with CAT2ndU and CATR as primers. During the PCR identification, popping-out URA3 marker gene after the second CAT allele was disrupted in C. tropicalis 02 by transformation of the gene disruption cassette CAT2-gda324-URA3-CAT2 were identified. All lanes showed strains with marker gene popped-out. The band with popped-out marker gene (CAT2-gda324-CAT2-fragment between CAT2 downstream homology arm and CAT1 downstream homology arm-CAT1) had a size of 1444 bp. The control used was PCR products with the C. tropicalis XZX chromosome as a template, with an original band (a CAT1 gene fragment between CAT2 upstream homology arm to CAT1 downstream homology arm) size of 1312 bp. The PCR primers were CAT2ndU/CATR. Marker gene pop-out was verified by sequencing. PCR identification results are shown in FIG. 17.

Sequencing of the PCR product CAT1-gda324-CAT1-CATLD at the CAT gene locus after the marker gene was popped out according to Example 1, revealed that there was fragment loss between the two CAT1 homology arms of a single CAT allele, and the lost fragment was substituted by a gda sequence. This was confirmed by carrying out a sequence comparison. Thus, it was verified at the molecular level that this single copy of the CAT sequence was disrupted, and it was also verified that in the process of pop-out of the marker gene, only the URA3 gene fragment between the two gda sequences having the same direction was popped out(in the two gda sequences, one gda sequence exists in the URA3 gene, the other one gda sequence is from the gene disruption cassette, the two gda sequences are exactly the same). Sequencing of the PCR product CAT2-gda324-CAT2-CAT1 at the CAT gene locus after the marker gene was popped out according to Example 8 showed that the sequence of the PCR product conformed with the sequence according to theoretical prediction (identity of the two sequences was 97.05%) and only the fragment between the two CAT2 homology arms was replaced by a gda fragment. Thus two-copy CAT allele disruption was further verified at the molecular level, showing that the gene disruption cassette of the used may be suitable for two-copy and multiple gene disruption of C. tropicalis.

TABLE 1
Comparison of recombination efficiency of gene disruption cassette of the present
invention and conventional gene disruption cassette

| | | | | Number of | |
|---|---|---|---|---|---|
| | | | | transformants | Recombination |
| | Total | Total no. | No. of | identified as | efficiency |
| Gene disruption | DNA | of | transformants | correct | (transformants/ |
| cassette | wt. (μg) | transformants | identified | transformation | μg DNA) |
| CAT1-gda143- | 7.27 | 31 | 24 | 11 | 1.95 |
| URA3-CAT1 | | | | | |
| CAT1-gda245- | 7.04 | 21 | 12 | 9 | 2.24 |
| URA3-CAT1 | | | | | |
| CAT1-gda324- | 4.98 | 17 | 11 | 4 | 1.24 |
| URA3-CAT1 | | | | | |
| CAT1-gda488- | 7.01 | 28 | 24 | 6 | 1.00 |
| URA3-CAT1 | | | | | |
| CAT1-His-URA3- | 15.51 | 49 | 48 | 2 | 0.13 |
| His-CAT1 | | | | | |

It can be seen from the statistical results shown in Table 1 that the transformation/recombination efficiency of the gene disruption cassette with gda143, gda245, gda324, gda488 used was greater by an order of magnitude than that of the gene disruption cassette of prior art (hisG-URA3-hisG).

TABLE 2
Effect of gda sequence length on URA3 gene pop-out efficiency

| | | | | No. | No. of | |
|---|---|---|---|---|---|---|
| | | | | Of | identified | |
| | | No. of | Total no. | iden- | detection | |
| | | colonies | of cells | tified | marker | |
| | gda | on | applied to | trans- | gene | |
| Ex. | size | FOA | FOA | form- | trans- | Detection |
| no. | (bp) | plate | plate | ants | formants | efficiency |
| 4 | 143 | 0, 1, 3 | 2.715 × 10⁹ | 4 | 3 | 3.7 × 10⁻¹⁰ |
| 3 | 245 | 0, 0, 2 | 2.86 × 10⁹ | 2 | 2 | 2.33 × 10⁻¹⁰ |
| 2 | 324 | 92, 99, | 2.055 × 10⁹ | 8 | 8 | 5.58 × 10⁻⁸ |
| | | 132 | | | | |
| 1 | 488 | 73, 82, | 5.395 × 10⁹ | 6 | 6 | 1.65 × 10⁻⁸ |
| | | 112 | | | | |
| 7 | 302 | 61, 72, | 5.16 × 10⁹ | 12 | 12 | 1.42 × 10⁻⁸ |
| | | 87 | | | | |
| 6 | 305 | 62, 99, | 5.84 × 10⁹ | 12 | 12 | 1.73 × 10⁻⁸ |
| | | 145 | | | | |
| 5 | 325 | 145, 146, | 2.33 × 10⁹ | 11 | 9 | 5.61 × 10⁻⁸ |
| | | 188 | | | | |
| comp. | hisG | 744, 711, | 9.43 × 10⁹ | 12 | 12 | 7.5 × 10⁻⁸ |
| ex. 1 | | 657 | | | | |

It can be seen from the statistical results shown in Table 2 that when the gda fragment length of the gene disruption cassette was 143 bp, the URA3 gene was efficiently popped out. When the gda fragment was longer than 300 bp, URA3 gene pop-out efficiency was markedly higher and was comparable to that of conventional HisG disruption cassettes, which further improved the overall efficiency of C. tropicalis gene disruption.

### REFERENCES

Irshad Ahmad, Woo Yong Shim et al. (2012). “Enhancement of xylitol production in C. tropicalis by co-expression of two genes involved in pentose phosphate pathway.” Bioprocess Biosyst Eng 35: 199-204.
Haas L, Cregg J et al. (1990). “Development of an integrative DNA transformation system for the yeast C. tropicalis.” Journal of Bacteriology 172 (8): 4571-4577.
Picataggio S, Deanda K et al. (1991). “Determination of C. tropicalis acyl coenzyme A oxidase isozyme function by sequential gene disruption.” Molecular and Cellular Biology 11 (9): 4333-4339.
Ko B S, Kim J et al. (2006). “Production of xylitol from D-xylose by a xylitol dehydrogenase gene-disrupted mutant of C. tropicalis.” Applied and Environmental Microbiology 2006, 72 (6): 4207-4213.
Gao Hong (2005). “Metabolic regulation of the β-oxidation pathway in the production of 1, 11-dicarboxylic acid through biocatalysis.” Beijing, Tsinghua University (Doctoral Dissertation).
Gong Yi, Jiang Hua et al. (1997). “Construction of new vector-host system in Candida tropicalis.” Chinese Journal of Biotechnology, 13 (3): 309-312.
Zheng Xiang, Xianzhong Chen et al. (2014). “Development of a genetic transformation system for C. tropicalis based on a reusable selection marker of URA3 gene.” Hereditas (Beijing) 10: 1053-1061.
Ueda T, Suzuki T et al. (1994). “Unique structure of new serine tRNAs responsible for decoding the leucine codon CUG in various Candida species and their putative ancestral tRNA genes.” Biochimie 76 (12): 1217-1222.
Alani E, Cao L, et al. (1987). “A method for gene disruption that allows repeated use of URA3 selection in the construction of multiply disrupted yeast strains.” Genetics. 1987 August; 116(4):541-5.R. Bryce Wilson, Dana Davis et al. (2000). “A recyclable Candida albicans URA3 cassette for PCR product-directed gene disruptions.” Yeast 16: 65-70.
Haas L, Cregg J et al. (1990) “Development of an integrative DNA transformation system for the yeast C. tropicalis.” Journal of Bacteriology 172 (8): 4571-4577.

1. A gene cassette for disruption of at least one target gene in a yeast cell, wherein the gene cassette comprises:
(a) a URA3 gene capable of being used as a marker gene;
(b) at least one gene disruption auxiliary (gda) sequence; and
(c) an upstream and a downstream sequences of the target gene,
wherein the gda sequence is from 300 to 600 bp in length and selected from within the nucleotide sequence of SEQ ID NO:39 and variants thereof.

2. The gene cassette according to claim 1, wherein (b) the gda sequence is from 300 to 500 bp in length.

3. The gene cassette according to claim 2, wherein (b) the gda sequence is selected from within the nucleotide sequence of SEQ ID NO:40.

4. The gene cassette according to claim 2, wherein (b) the gda sequence is selected from within the nucleotide sequence of SEQ ID NO:41.

5. The gene cassette according to claim 2, wherein (b) the gda sequence is selected from within the nucleotide sequence of SEQ ID NO: 42.

6. The gene cassette according to claim 2, wherein (b) the gda sequence is selected from within the nucleotide sequence of SEQ ID NO:43.

7. The gene cassette according to claim 1, wherein (b) the gda sequence is at least one nucleotide sequence selected from the group consisting of SEQ ID NOs: 16, 14 18 21 and 24.

8. The gene cassette according to claim 1, wherein the yeast cell is selected from the group consisting of Candida albicans, Candida tropicalis, Candida parapsilopsis, Candida krusei, Cryptococcus neoformans, Hansenular polymorpha, Issatchenkia orientalis, Kluyverei lactis, Kluyveromyces lactis, Kluyveromyces marxianus, Pichia pastoris, Saccharomyces cerevisiae, Schizosaccharomyces pombe, and Yarrowia lipolytica.

9. The gene cassette according to claim 1, wherein the yeast cell is uracil auxotrophic C. tropicalis.

10. The gene cassette according to claim 1, wherein (a) the URA3 gene comprises the nucleotide sequence of SEQ ID NO:3.

11. The gene cassette according to claim 1, wherein (c) the upstream and downstream sequences of the target gene are each ≥50 bp in length.

12. A method of disrupting the expression of at least one target gene in at least one yeast cell, the method comprises transforming the yeast cell with at least one vector comprising the gene cassette according to claim 1.

13. The method of claim 12, wherein the yeast cell is uracil auxotrophic C. tropicalis.

14. The method according to claim 12, wherein the gda sequence is at least one nucleotide sequence selected from the group consisting of SEQ ID NOs: 16, 14, 18, 21 and 24.

15. A genetically modified yeast cell comprising a gene cassette according to claim 1.


SEQUENCE LISTING (43 sequences)

SEQ ID NO: 1 - DNA, 21 bp, Artificial Sequence
  URAU primer
tactctaacgacgggtacaac

SEQ ID NO: 2 - DNA, 21 bp, Artificial Sequence
  URAR primer
acccgatttcaaaagtgcaga

SEQ ID NO: 3 - DNA, 1578 bp, Candida tropicalis
tctaacgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaaaaaaaaagtgaaaaaaaaaatcaaacccaaagatcaacctccccatcatcatcgtcatcaaacccccagctcaattcgcaatggttagcacaaaaacatacacagaaagggcatcagcacacccctccaaggttgcccaacgtttattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctgacccttgagcttcgcctcgttgtaatgattatacacatccaacgcttccaacctcgataaatggatcttctgcacttttgaaatcgggt

SEQ ID NO: 4 - DNA, 25 bp, Candida tropicalis
aactgcagttctgactggtaccgat

SEQ ID NO: 5 - DNA, 29 bp, Artificial Sequence
  Ugda primer
gcgtcgacacccgatttcaaaagtgcaga

SEQ ID NO: 6 - DNA, 488 bp, Candida tropicalis
ttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctgacccttgagcttcgcctcgttgtaatgattatacacatccaacgcttccaacctcgataaatggatcttctgcacttttgaaatcgggt

SEQ ID NO: 7 - DNA, 20 bp, Artificial Sequence
  CATU primer
gtttaactttaagttgtcgc

SEQ ID NO: 8 - DNA, 21 bp, Artificial Sequence
  CATR primer
tacaacttaggcttagcatca

SEQ ID NO: 9 - DNA, 1881 bp, Candida tropicalis
gtttaactttaagttgtcgcaacaagtattaaagaattccaccaaatccattatgccaattttgaaaaaaccattctccaccagccacgcaaagggtgacttgttcaaataccagtcacaattacccaagttgcctgttcctactttggaagaaaccgcatccaagtacctcaagaccgttgagccattcttgaaccaagagcaattggaatccaccaaggccaaagtcgctgagtttgttagaccaggtggtgccggtgaagccttgcaagccagattgaacaactttgccgccgacaaggacaactggttggctgaattttgggacgactatgcatacatgtcttatagagatcctgttgttccatatgtttcttactttttcagtcacaaggatgtcaagaacatcattggccaagaccaattgttgaaggccactttgattgcttactacactattgagttccaagaaaaggttttggacgaaagtttggacccagaagtcatcaagggtaacccattctgtatgaacgccttcaagtacatgttcaacaactcgagagttccagctgaaggctccgacatcacccaacactacaacggtgaagaaaaccaatttttcgttgtcatctacaagaacaacttctacaaggttccaacccacaagaacggccaaagattgaccaagggtgaaatctacagctacttgcaagaaatcaagaacgatgccactccaaagggtctcggtttgggtgctttgacctcattgaacagagacgaatggttgagtgcctacaacaacttgttgaagtccccaatcaacgaagcttccttgggatccatctttgcttccagctttgtcattgccttggactccaacaacccagtcaccattgaagaaaaatccaagaactgctggcacggggacggtcaaaacagattctttgacaagcctttggaattcttcgtcagtgctaacggtaactctggtttccttggtgaacactccagaatggacgctaccccaaccgtgcaattgaacaacaccatctacaagcaaatcttggaaaccaatccaaacgacttgattgttgaaattggttcttctgctccaagattcggcaatgctgaaatcttgcctttcgacatcaacccaaccaccagagccaacatcaaagacgctattgccaagtttgacgccaccattgctgcccacgacgaagaaatcttccaacactacggttacggtaagggattgatcaagaagttcaaggtctccccagatgcctacgtgcaattgttgatgcaattggcatacttcaagtacaccggcaagatcagaccaacttatgaatccgccgccaccagaaagttcttgaagggtagaaccgaaaccggtagaactgtttccaacgaatccaagaagtttgttgagacctggtccgatccaaacgctagcagcgccgacaaggttgccactttccaagctgccgctaagcaacacgttgcttacttgtctgctgccgccgatggtaagggtgtcgaccgtcacttgtttggtttgaagcagatgattcaaccaggcgaaccaatccctgaaatcttcactgacccaatctttagctattctcaaacctggtacatttcttcttcccaagtcccatctgaattcttccaatcttggggttggtcgcaagtcattgacgacggtttcggtttggcttacttgatcaacaacgactggatccacgttcacatttcttgtaagagaggtaacggcttgcaatccgaccacttgaaatggtacttggttgatagtgctaacgaaatgaaggatgtcttgactaagggattattgactgatgctaagcctaagttgta

SEQ ID NO: 10 - DNA, 28 bp, Artificial Sequence
  rCATU primer
aactgcagccaaaattcagccaaccagt

SEQ ID NO: 11 - DNA, 29 bp, Artificial Sequence
  rCATR primer
gctctagaagatgattcaaccaggcgaac

SEQ ID NO: 12 - DNA, 21 bp, Artificial Sequence
  CATLD primer
aatagaaactagcaatcggaa

SEQ ID NO: 13 - DNA, 29 bp, Artificial Sequence
  Ugda324 primer
aactgcagactaagcttctaggacgtcat

SEQ ID NO: 14 - DNA, 324 bp, Candida tropicalis
actaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctgacccttgagcttcgcctcgttgtaatgattatacacatccaacgcttccaacctcgataaatggatcttctgcacttttgaaatcgggt

SEQ ID NO: 15 - DNA, 29 bp, Artificial Sequence
  Ugda245 primer
aactgcagaatggatgtagcagggatggt

SEQ ID NO: 16 - DNA, 245 bp, Candida tropicalis
aatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctgacccttgagcttcgcctcgttgtaatgattatacacatccaacgcttccaacctcgataaatggatcttctgcacttttgaaatcgggt

SEQ ID NO: 17 - DNA, 28 bp, Artificial Sequence
  Ugda143 primer
aactgcagtgcttgaaggtatccacgta

SEQ ID NO: 18 - DNA, 143 bp, Candida tropicalis
cttgaaggtatccacgtacgagttgtaatacaccttgaagaacggcttcgtctgacccttgagcttcgcctcgttgtaatgattatacacatccaacgcttccaacctcgataaatggatcttctgcacttttgaaatcgggt

SEQ ID NO: 19 - DNA, 27 bp, Artificial Sequence
  Ugda325 primer
aactgcagtcgtgattgggttcatcgc

SEQ ID NO: 20 - DNA, 26 bp, Artificial Sequence
  Dgda325 primer
gcgtcgaccaatgacgtcctagaagc

SEQ ID NO: 21 - DNA, 325 bp, Candida tropicalis
tcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattg

SEQ ID NO: 22 - DNA, 28 bp, Artificial Sequence
  Ugda305 primer
gctctagatctaacgacgggtacaacga

SEQ ID NO: 23 - DNA, 27 bp, Artificial Sequence
  Dgda305 primier
cggaattcacgtgactagtatggcaat

SEQ ID NO: 24 - DNA, 305 bp, Candida tropicalis
tctaacgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgt

SEQ ID NO: 25 - DNA, 27 bp, Artificial Sequence
  Ugda302 primer
gctctagacatacacagaaagggcatc

SEQ ID NO: 26 - DNA, 25 bp, Artificial Sequence
  Dgda302 primer
cggaattcgtactgcaacatcacgg

SEQ ID NO: 27 - DNA, 302 bp, Candida tropicalis
catacacagaaagggcatcagcacacccctccaaggttgcccaacgtttattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtac

SEQ ID NO: 28 - DNA, 29 bp, Artificial Sequence
  HisG-F1 primer
ccggaattcttccagtggtgcatgaacgc

SEQ ID NO: 29 - DNA, 30 bp, Artificial Sequence
  HisG-R1 primer
cgcggatccgctgttccagtcaatcagggt

SEQ ID NO: 30 - DNA, 30 bp, Artificial Sequence
  HisG-F2 primer
acgcgtcgacttccagtggtgcatgaacgc

SEQ ID NO: 31 - DNA, 29 bp, Artificial Sequence
  HisG-R2 primer
aactgcaggctgttccagtcaatcagggt

SEQ ID NO: 32 - DNA, 1157 bp, Salmonella typhimurium
ggatccgctgttccagtcaatcagggtattgaagctcatggtctttactccatcacagggttccgccttatccggcctactgaacccaaaatatcaacgcattacgtaggcctgataagcgcagcgccatcaggcgtcagatcactccatcatcttctcgatcggcagtaccagaatcgagctggcgccaagcgctttcagtttctccatggtttcccagaacaacgtttcgctgctgaccatgtgcatcgccacgcgctgttgctcgcctgccagcggcagaattgtcggcctttcggcgcctggcagcagggcgataacctcttccagtcgttcacttggcgcgtgcatcatgatgtatttcgattcgcgcgcctgaatcacgccctgaatacgggtcagcaatttatcgatcagctgttgcttgctctgtgccatctcaccgtcgcgctgaatcagacaggctttagagcggtagataacttcgacttcacgcaggccgttagcttcaagcgtcgcgccggtagagaccaaatcgcagatagcgtcggccagccccgcgcgcggcgcgacttcgacagaaccatttaacagacacgatttaaaagagacgcctttctggtcgaggtagcgtttgaggaggtgcggataagaggtagcgatacgtttaccgtccagcgcggccgggccgtcccaggcttcgtcaaccggcgttgccagcgataaacggcagccgccgaagtcaagacggcgcagggttaaatagcgtggatcttcgccctgcgcgcggcggttgagtagctcttcttccagcacgttttcgccgataataccgagatcgaccacgccatccatcaccagacccggaatgtcatcatcacgcacgcgcaggatatcaatcggcatgttttccgccatcgcaatcaggcgctgagtgtgtaaattaatttttatgccgcagcgggccagcaattctcgtgaatcatcgcttaaacggcctgatttctgaatagctatgcgtaagcgggtgttgtctaacattctgcgttcctctttatcctgtctgaaccggtctgtatcgcgcgccaaaaaaaaagcccccggaagatgatcttccgggggctttctcatgcgttcatgcaccactggaagaattc

SEQ ID NO: 33 - DNA, 1157 bp, Salmonella typhimurium
ctgcaggctgttccagtcaatcagggtattgaagctcatggtctttactccatcacagggttccgccttatccggcctactgaacccaaaatatcaacgcattacgtaggcctgataagcgcagcgccatcaggcgtcagatcactccatcatcttctcgatcggcagtaccagaatcgagctggcgccaagcgctttcagtttctccatggtttcccagaacaacgtttcgctgctgaccatgtgcatcgccacgcgctgttgctcgcctgccagcggcagaattgtcggcctttcggcgcctggcagcagggcgataacctcttccagtcgttcacttggcgcgtgcatcatgatgtatttcgattcgcgcgcctgaatcacgccctgaatacgggtcagcaatttatcgatcagctgttgcttgctctgtgccatctcaccgtcgcgctgaatcagacaggctttagagcggtagataacttcgacttcacgcaggccgttagcttcaagcgtcgcgccggtagagaccaaatcgcagatagcgtcggccagccccgcgcgcggcgcgacttcgacagaaccatttaacagacacgatttaaaagagacgcctttctggtcgaggtagcgtttgaggaggtgcggataagaggtagcgatacgtttaccgtccagcgcggccgggccgtcccaggcttcgtcaaccggcgttgccagcgataaacggcagccgccgaagtcaagacggcgcagggttaaatagcgtggatcttcgccctgcgcgcggcggttgagtagctcttcttccagcacgttttcgccgataataccgagatcgaccacgccatccatcaccagacccggaatgtcatcatcacgcacgcgcaggatatcaatcggcatgttttccgccatcgcaatcaggcgctgagtgtgtaaattaatttttatgccgcagcgggccagcaattctcgtgaatcatcgcttaaacggcctgatttctgaatagctatgcgtaagcgggtgttgtctaacattctgcgttcctctttatcctgtctgaaccggtctgtatcgcgcgccaaaaaaaaagcccccggaagatgatcttccgggggctttctcatgcgttcatgcaccactggaagtcgac

SEQ ID NO: 34 - DNA, 20 bp, Artificial Sequence
  CAT2ndU primer
ctgaaggctccgacatcacc

SEQ ID NO: 35 - DNA, 20 bp, Artificial Sequence
  CAT2ndR primer
caaccttgtcggcgctgcta

SEQ ID NO: 36 - DNA, 906 bp, Candida tropicalis
ctgaaggctccgacatcacccaacactacaacggtgaagaaaaccaatttttcgttgtcatctacaagaacaacttctacaaggttccaacccacaagaacggccaaagattgaccaagggtgaaatctacagctacttgcaagaaatcaagaacgatgccactccaaagggtctcggtttgggtgctttgacctcattgaacagagacgaatggttgagtgcctacaacaacttgttgaagtccccaatcaacgaagcttccttgggatccatctttgcttccagctttgtcattgccttggactccaacaacccagtcaccattgaagaaaaatccaagaactgctggcacggggacggtcaaaacagattctttgacaagcctttggaattcttcgtcagtgctaacggtaactctggtttccttggtgaacactccagaatggacgctaccccaaccgtgcaattgaacaacaccatctacaagcaaatcttggaaaccaatccaaacgacttgattgttgaaattggttcttctgctccaagattcggcaatgctgaaatcttgcctttcgacatcaacccaaccaccagagccaacatcaaagacgctattgccaagtttgacgccaccattgctgcccacgacgaagaaatcttccaacactacggttacggtaagggattgatcaagaagttcaaggtctccccagatgcctacgtgcaattgttgatgcaattggcatacttcaagtacaccggcaagatcagaccaacttatgaatccgccgccaccagaaagttcttgaagggtagaaccgaaaccggtagaactgtttccaacgaatccaagaagtttgttgagacctggtccgatccaaacgctagcagcgccgacaaggttg

SEQ ID NO: 37 - DNA, 30 bp, Artificial Sequence
  rCAT2ndU primer
aactgcagatctgttttgaccgtccccgtg

SEQ ID NO: 38 - DNA, 30 bp, Artificial Sequence
  rCAT2ndR primer
gctctagatcttgcctttcgacatcaaccc

SEQ ID NO: 39 - DNA, 1578 bp, Candida tropicalis; identical to SEQ ID NO: 3

SEQ ID NO: 40 - DNA, 738 bp, Candida tropicalis
tctaacgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaaaaaaaaagtgaaaaaaaaaatcaaacccaaagatcaacctccccatcatcatcgtcatcaaacccccagctcaattcgcaatggttagcacaaaaacatacacagaaagggcatcagcacacccctccaaggttgcccaacgtttattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtac

SEQ ID NO: 41 - DNA, 626 bp, Candida tropicalis
tcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctgacccttgagcttcgcctcgttgtaatgattatacacatccaacgcttccaacctcgataaatggatcttctgcacttttgaaatcgggt

SEQ ID NO: 42 - DNA, 354 bp, Candida tropicalis
atattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctgacccttgagcttcgcctcgttgtaatgattatacacatccaacgcttccaacctcgataaatggatcttctgcacttttgaaatcgggt

SEQ ID NO: 43 - DNA, 360 bp, Candida tropicalis
atggttagcacaaaaacatacacagaaagggcatcagcacacccctccaaggttgcccaacgtttattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacg